SEQUENCE LISTING (19 sequences)

SEQ ID NO: 1 - DNA, 1509 bp, Serratia marcescens
atgcaatctactaaaaaggcaattgaaattactgaatccagccttgcggccgcgacaaccggctacgatgctgtagatgacctgctgcattatcatgagcggggtaacgggattcagattaatggcaaggattcattttctaacgagcaagctgggctgtttattacccgcgagaaccaaacctggaacggttacaaggtatttggccagccggtcaaattaaccttctccttcccggactataagttctcttccaccaacgtcgccggcgataccgggctgagcaagttcagcgcggaacagcagcagcaggctaagctgtcgctgcagtcctgggccgacgtcgccaatatcaccttcaccgaagtggcggccggtcaaaaggccaatatcaccttcggcaattacagccaggatcgtcccggccactatgattatggtacccaggcctacgccttcctgccgaacaccatttggcagggccaggatttgggcggccagacctggtacaacgtcaaccaatccaacgtgaagcatccggcgaccgaagactacggccgccagacgttcacccatgagattggccatgcgctgggcctgagccacccgggcgactacaacgccggtgagggcaacccgacctataacgacgttacctatgcggaagatacccgccagttcagcctgatgagctactggagtgaaaccaacaccggtggcgacaacggcggtcactatgccgcggctccgctgctggatgacattgccgccattcagcatctgtatggcgcgaacctgtcgacccgcaccggcgacaccgtgtacggctttaactccaataccggtcgtgacttcctcagcaccaccagcaattcgcagaaagtgatctttgcggcctgggatgcgggtggcaacgataccttcgacttctccggttataccgctaaccagcgcatcaacctgaatgagaaatcgttctccgacgtgggcggcctgaagggcaacgtctcgatcgccgccggtgtgaccattgagaacgccattggcggttccggcaacgacgtgatcgtcggcaacgcggccaacaacgtgctgaaaggcggcgcgggtaacgacgtgctgttcggcggcggcggggcggatgaactgtggggcggtgccggcaaagacatctttgtgttctctgccgccagcgattccgcaccgggtgcttccgactggatccgcgacttccagaaagggatcgacaagatcgacctgtcgttcttcaataaagaagcgcagagcagcgatttcattcacttcgtcgatcacttcagcggcacggccggtgaggcgctgctgagctacaacgcgtccagcaacgtgaccgatttgtcggtgaacatcggcgggcatcaggcgccggacttcctggtgaaaatcgtcggccaggtagacgtcgccacggactttatcgtgtaacacagcaacggaacgcccggcgcagtctcggccgggcgtgatgca

SEQ ID NO: 2 - protein, 502 aa, Serratia marcescens
MQSTKKAIEITESSLAAATTGYDAVDDLLHYHERGNGIQINGKDSFSNEQAGLFITRENQTWNGYKVFGQPVKLTFSFPDYKFSSTNVAGDTGLSKFSAEQQQQAKLSLQSWADVANITFTEVAAGQKANITFGNYSQDRPGHYDYGTQAYAFLPNTIWQGQDLGGQTWYNVNQSNVKHPATEDYGRQTFTHEIGHALGLSHPGDYNAGEGNPTYNDVTYAEDTRQFSLMSYWSETNTGGDNGGHYAAAPLLDDIAAIQHLYGANLSTRTGDTVYGFNSNTGRDFLSTTSNSQKVIFAAWDAGGNDTFDFSGYTANQRINLNEKSFSDVGGLKGNVSIAAGVTIENAIGGSGNDVIVGNAANNVLKGGAGNDVLFGGGGADELWGGAGKDIFVFSAASDSAPGASDWIRDFQKGIDKIDLSFFNKEAQSSDFIHFVDHFSGTAGEALLSYNASSNVTDLSVNIGGHQAPDFLVKIVGQVDVATDFIVHSNGTPGAVSAGRDA

SEQ ID NO: 3 - DNA, 416 bp, Serratia marcescens
atgaaaggtactttagcgcgcaccgctttggcggcgggtggcatgatggtgacgagtgcggtgatggccggcagtttggcattgccgaccgcgcagtcgctggcggggcaatggcaggtggccgacagcgaacggcaatgccaaatcgagtttctggcgcatgaacaaaacgagaccaacggctatcagctggtggatcggcaacagtgtttgcagagcgtgtttgcggcggaagtcgtgggctggcgcccggctccggacggcatcgccttgctgcgggcggatggcagcacgctggcgttcttctcgcgcgacggcgatctgtaccgcaatcagctgggtgcggatgatgctctgacgttgaaagcgctggcttgatgaagaaacgggttcggcgcgccgaacccgcggtaggt

SEQ ID NO: 4 - protein, 135 aa, Serratia marcescens
MKGTLARTALAAGGMMVTSAVMAGSLALPTAQSLAGQWQVADSERQCQIEFLAHEQNETNGYQLVDRQQCLQSVFAAEVVGWRPAPDGIALLRADGSTLAFFSRDGDLYRNQLGADDALTLKALARNGFGAPNPR

SEQ ID NO: 5 - DNA, 1464 bp, Serratia marcescens
  misc_feature: Genebank accession no. X55521
atgcaatctactaaaaaggcaattgaaattactgaatccagccttgcggccgcgacaaccggctacgatgctgtagacgacctgctgcattatcatgagcggggtaacgggattcagattaatggcaaggattcattttctaacgagcaagctgggctgtttattacccgcgagaaccaaacctggaacggttacaaggtatttggccagccggtcaaattaaccttctccttcccggactataagttctcttccaccaacgtcgccggcgacaccgggctgagcaagttcagcgcggaacagcagcagcaggctaagctgtcgctgcagtcctgggccgacgttgccaatatcaccttcaccgaagtggcggccggtcaaaaggccaatatcaccttcggcaattacagccaggatcgtcccggccactatgattatggtacccaggcctacgccttcctgccgaacaccatttggcagggccaggatttgggcggccagacctggtataacgtcaaccaatccaacgtgaagcatccggcgaccgaagactacggccgccagacgttcacccatgagattggccatgcgctgggcctgagccacccgggcgactacaacgccggtgagggcaacccgacctataacgacgtcacctatgcggaagatacccgccagttcagcctgatgagctactggagtgaaaccaacaccggtggcgacaacggcggtcactatgccgcggctccgttgctggatgacattgccgccattcagcatctgtatggcgccaacccgtcgacccgcaccggcgacaccgtgtacggctttaactccaacaccggtcgtgacttcctcagcaccaccagcaattcgcagaaagtgatctttgcggcctgggatgcgggtggcaacgataccttcgacttctccggttataccgctaaccagcgcatcaacctgaatgagaaatcgttctccgacgtgggcggcctgaagggcaacgtctcgatagccgccggtgtgaccattgagaacgccattggcggttccggcaatgacgtgatcgtcggcaacgcggccaacaacgtgctgaaaggcggcgcgggtaacgacgtgctgttcggcggcggcggggcggatgaactgtggggcggtgccggcaaagacatctttgtgttctctgccgccagcgattccgcaccgggtgcttccgactggatccgcgacttccagaaagggatcgacaagatcgacctgtcgttcttcaataaagaagcgaatagcagtgatttcatccacttcgtcgatcacttcagcggcacggccggtgaggcgctgctgagctacaacgcgtccagcaacgtgaccgatttgtcggtgaacatcggcgggcatcaggcgccggacttcctggtgaaaatcgtcggccaggtagacgtcgccacggactttatcgtgtaa

SEQ ID NO: 6 - DNA, 1461 bp, Serratia marcescens
  misc_feature: Genbank accession no. X04127
atgcaatctactaaaaaggcaattgaaattactgaatccaacttcgcggccgccacaaccggctacgatgctgtagacgacctgttgcattatcatgagcggggcaacgggattcagattaatggcaaggattcattttctaacgagcaagctgggctgtttattacccgtgagaaccaaacctggaacggttacaaggtatttggccagccggtcaaattaaccttctccttcccggactataagttctcttccaccaacgtcgccggcgacaccgggctgagcaagttcagcgcggaacagcagcagcaggctaagctgtcgctgcagtcctgggccgacgtcgccaatatcacctttaccgaagtggcggccggacaaaaggccaacatcaccttcggtaactacagccaggatcgtcccggccactatgattacggcacccaggcctacgccttcctgccgaacaccatttggcaggggcaggatctggggggccagacctggtacaacgtcaaccagtccaacgtgaagcatccggcgaccgaagactacggccgccagacgtttacccatgagattggccatgcgctgggtctgagccatccgggcgattacaacgccggtgaaggcaacccgacctatcgcgacgtcacttatgcggaagacacccgtcagttcagcctgatgagctactggagcgaaaccaacaccggtggtgataacggcggtcattacgccgcagctccgctgctggatgacattgccgccattcaacatctgtatggcgccaacctgtcgacccgcaccggcgacaccgtgtacggttttaactccaacaccggtcgtgacttcctcagcaccaccagcaattcgcagaaagtgatctttgcggcctgggatgcgggtggcaacgataccttcgacttctccggttataccgctaaccagcgcatcaacctgaacgagaagtcgttctccgacgtgggcggcctgaaaggcaacgtctcgatcgccgccggtgtgaccatcgagaacgccattggcttccggcaacgactgatcgtcggcaacgcggccaataacgtgctgaaaggcggcgcgggtaacgacgtgctgttcggcggcggcggggcggatgagctgtggggcggtgccggtaaagacatcttcgtgttctctgccgccagcgattccgcgccgggcgcttcagactggatccgcgacttccagaaagggatcgacaagattgatctttcgttcttcaacaaagaagcgcagagcagcgatttcattcacttcgtcgatcacttcagcggcgcggccggtgaagcgctgctgagctacaacgcgtccaacaacgtgaccgatttgtcggtgaacatcggtggtcatcaggcgcctgacttcctggtgaaaatcgtcggtcaggtagacgtcgccactgactttatcgtgtaa

SEQ ID NO: 7 - protein, 487 aa, Serratia marcescens
  MISC_FEATURE: Genebank accession no. X55521
MQSTKKAIEITESSLAAATTGYDAVDDLLHYHERGNGIQINGKDSFSNEQAGLFITRENQTWNGYKVFGQPVKLTFSFPDYKFSSTNVAGDTGLSKFSAEQQQQAKLSLQSWADVANITFTEVAAGQKANITFGNYSQDRPGHYDYGTQAYAFLPNTIWQGQDLGGQTWYNVNQSNVKHPATEDYGRQTFTHEIGHALGLSHPGDYNAGEGNPTYNDVTYAEDTRQFSLMSYWSETNTGGDNGGHYAAAPLLDDIAAIQHLYGANPSTRTGDTVYGFNSNTGRDFLSTTSNSQKVIFAAWDAGGNDTFDFSGYTANQRINLNEKSFSDVGGLKGNVSIAAGVTIENAIGGSGNDVIVGNAANNVLKGGAGNDVLFGGGGADELWGGAGKDIFVFSAASDSAPGASDWIRDFQKGIDKIDLSFFNKEANSSDFIHFVDHFSGTAGEALLSYNASSNVTDLSVNIGGHQAPDFLVKIVGQVDVATDFIV

SEQ ID NO: 8 - protein, 486 aa, Serratia marcescens
  MISC_FEATURE: Genbank accession no. X04127
MQSTKKAIEITESNFAAATTGYDAVDDLLHYHERGNGIQINGKDSFSNEQAGLFITRENQTWNGYKVFGQPVKLTFSFPDYKFSSTNVAGDTGLSKFSAEQQQQAKLSLQSWADVANITFTEVAAGQKANITFGNYSQDRPGHYDYGTQAYAFLPNTIWQGQDLGGQTWYNVNQSNVKHPATEDYGRQTFTHEIGHALGLSHPGDYNAGEGNPTYRDVTYAEDTRQFSLMSYWSETNTGGDNGGHYAAAPLLDDIAAIQHLYGANLSTRTGDTVYGFNSNTGRDFLSTTSNSQKVIFAAWDAGGNDTFDFSGYTANQRINLNEKSFSDVGGLKGNVSIAAGVTIENAIGFRQRLIVGNAANNVLKGGAGNDVLFGGGGADELWGGAGKDIFVFSAASDSAPGASDWIRDFQKGIDKIDLSFFNKEAQSSDFIHFVDHFSGAAGEALLSYNASNNVTDLSVNIGGHQAPDFLVKIVGQVDVATDFIV

SEQ ID NO: 9 - DNA, 379 bp, Serratia marcescens
  misc_feature: Genbank accession no. X55521
atgaaaggtactttagcacgcaccgctttggcggcgggtggcatgatggtgacgagtgcggtgatggccggcagtttggcattgccgaccgcgcagtcgctggcgggacaatggcaggtggccgacagcgaacggcaatgccaaatcgagtttctggcgcatgaacaaagcgagaccaacggctatcagctggtggatcggcaacagtgtttgcagagcgtgtttgcggcggaagtcgtgggctggcgcccggctccggacggcatcgccctgctgcgggcggatggcagcacgctggcgttcttctcgcgcgaggcatctgtaccgcaatcagctgggtgcggatgatgctttgacgttgaaagcgctggcttgatga

SEQ ID NO: 10 - DNA, 378 bp, Serratia marcescens
  misc_feature: Genbank accession no. L09107
atgaaaggtactttaacgcgcgccgccctggcggcgggaggcatgatggtgacgagtgcggtgatggccggcagtctggcgctgccgaccgcgcagtcgctggcggggcaatgggaggtagccgacagcgaacggcaatgccaaatcgagtttctggccaatgagcaaagcgagaccaacggctaccagctggtggatcggcagcgttgtctgcaaagcgtatttgcggccgaggtggtggctggcgcggggccggacggcatcgccctgctgcaggcggatggcagcacgctggcgttcttctcgcgcgacggcgatctataccgcaatcagctgggtgcgggtgatgccctgacgttgaaagcgctggcttgatga

SEQ ID NO: 11 - protein, 124 aa, Serratia marcescens
  MISC_FEATURE: Genbank accession no. L09107
MKGTLTRAALAAGGMMVTSAVMAGSLALPTAQSLAGQWEVADSERQCQIEFLANEQSETNGYQLVDRQRCLQSVFAAEVVAGAGPDGIALLQADGSTLAFFSRDGDLYRNQLGAGDALTLKALA

SEQ ID NO: 12 - DNA, 26 bp, Serratia marcescens
gcttacggggaggttatgtctatctc

SEQ ID NO: 13 - DNA, 31 bp, Serratia marcescens
ggtagagctcgtgcgtgctaaagtacctttc

SEQ ID NO: 14 - DNA, 26 bp, Serratia marcescens
gcttacggggaggttatgtctatctc

SEQ ID NO: 15 - DNA, 31 bp, Serratia marcescens; identical to SEQ ID NO: 13

SEQ ID NO: 16 - DNA, 23 bp, Serratia marcescens
ggccactatgattatggtaccca

SEQ ID NO: 17 - DNA, 20 bp, Serratia marcescens
gttataccgctaaccagcgc

SEQ ID NO: 18 - DNA, 27 bp, Serratia marcescens
gtgaaaatcgtcggccaggtagacgtc

SEQ ID NO: 19 - DNA, 28 bp, Serratia marcescens
ggctttctgttacttctccggccacttc

(19) United States
(12) Patent Application Publication
(10) Pub. No.: US 2004/0185566 A1
(43) Pub. Date: Sep. 23, 2004

(54) Polypeptides toxic to nematodes and methods of use

(76) Inventor: Peter Salamone, West Des Moines, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/392,432

(22) Filed: Mar. 19, 2003

(51) Int. Cl. C12N 15/82; C07H 21/04; C12N 15/87; C12N 15/09; C12N 15/63; C07K 1/00; C07K 14/00; C07K 16/00; C12N 5/04; C12N 5/10

(52) U.S. Cl. 435/468; 536/23.6; 435/320.1; 435/419; 435/471; 435/412; 800/278; 530/350; 800/279; 530/387.1

(57) ABSTRACT

The present invention provides polynucleotides and related polypeptides of a newly discovered serratia metalloprotease (SMP), as well as polynucleotides and related polypeptides of a newly discovered serratia metalloprotease inhibitor (SmaPI). The present invention further relates broadly to the use of the newly discovered SMP as a nematocidal agent, either as an administered agent, or as part of an integrated management system. The polynucleotides can be used to transform plants normally susceptible to free-living or burrowing plant parasitic nematodes, such as those of the genera Ascaris and Radopholus.

### BACKGROUND OF THE INVENTION

The present invention relates to the control of nematode pests.

There are nematode parasites of plants and animals, including humans. Nematodes (nema—thread; oides—resembling), which are unsegmented roundworms with elongated, fusiform, or saclike bodies covered with cuticle, are virtually ubiquitous in nature, inhabiting soil, water and plants, and are importantly involved in a wide range of animal and plant parasitic diseases. The plant parasites can cause significant economic losses in sub-tropical, tropical and temperate agriculture. Plant-parasitic nematodes are small (generally 100-300 μm long but up to 4 mm long, and 15-35 μm wide) worm-like animals which feed on root, stem or leaf tissues of living plants.

In general, nematodes are present wherever plants are cultivated. Ectoparasitic nematodes, such as the dagger (Xiphinema and Longidorus spp.), stubby-root (Trichodorus and Paratrichodorus spp.) and spiral (Scutellonema and Helicotylenchus spp.) nematodes, live outside the plant and pierce the plant cells with their stylet in order to feed. Migratory endoparasitic nematodes, such as the lesion (Pratylenchus spp.), stem and bulb (Ditylenchus spp.) and burrowing (Radopholus spp.) nematodes, live and feed inside the plant, migrating through the plant tissues. Sedentary endoparasitic nematodes, such as the root-knot (Meloidogyne spp.), cyst (Globodera and Heterodera spp.), citrus (Tylenchulus spp.) and reniform (Rotylenchulus spp.) nematodes, live and feed inside the plant, inducing specialized fixed feeding sites called giant cells, syncytia or nurse cells in susceptible plants. Such fixed feeding sites serve as food transfer cells for the various developmental stages of the nematodes. Syncytia originate in the pericycle, endodermis or adjacent cortex.

Ascaris species are the largest nematodes that infect human (Ascaris lumbricoides) and swine (Ascaris suum) hosts. The Ascaris species produce protease inhibitors that are believed to be a defense mechanism that enables the nematode to survive hostile environments of the host intestine (Peeters 1976).

Efforts to control nematodes have led to combinations of greater and greater toxicity and expense. Even the marginally effective current therapies for both plant and animals are so environmentally harmful and personally hazardous, that most previously available treatments have been banned or are slated for worldwide termination in the future.

Various methods have been used to control plant parasitic nematodes. They include quarantine measures, manipulation of planting and harvesting dates, improved fertilization and irrigation programs that lessen plant stresses, crop rotation and following, use of resistant and tolerant cultivars and rootstocks, organic soil amendments, and physical (e.g., solarization), biological and chemical control. Although quarantines are useful, especially when an infestation is first discovered, they are very expensive measures and usually cannot prevent the spread of nematodes. Furthermore, biological control is difficult to manage, and high quantities and repeated additions of agents are required.

Currently, control of plant-parasitic nematodes relies mainly on chemical control. Nematocides used commercially are generally either fumigants (e.g., halogenated aliphatic hydrocarbons and methyl isothiocyanate precursor compounds) or non-fumigants (e.g., organophosphates and oximecarbamates). However, the use of chemical nematocides is undesirable because these chemicals are highly toxic and therefore present a hazard to the user and to the environment.

In spite of the destruction nematodes cause, there has been little progress made in their control. This is partially due to the anatomy of a nematode, which has evolved over generations to be impervious to elements and to withstand harsh environments.

The cuticle of nematodes is a thin, flexible outer covering that is morphologically similar in both free-living and parasitic species (Fetterer and Rhodes 1993). The cuticle is composed of a series of layers that differ in composition. The innermost are the median and basal layers. These are composed mostly of collagenous proteins. A series of struts connect the median layer and the cortical layer. The internal cortical layer is predominantly collagenous. The external cortical layer is the epicuticle (Fetterer and Rhodes 1993; Johnstone 1994). A highly insoluble and fibrous protein is a major component of the external corticle layer and perhaps the epicuticle (Fujimoto and Kanaya 1973).

Structural studies of the cuticle indicate that it is primarily composed of protein with only trace amounts of lipid and carbohydrate. Although the cuticle has a complex structure, only three general categories of proteins have been identified: (1) collagenous proteins that can be solubilized from the cuticle with reducing agents; (2) non-collagenous proteins that remain insoluble in the presence of reducing agents, detergents, or salts; and (3) surface associated non-collagenous proteins that are soluble in the presence of detergents or salts (Fetterer and Rhodes 1993).

Collagen is the major structural component of a nematodes cuticle. In the Ascaris species, cuticle collagens are a part of a large multigene family that code for monomer polypeptides of approximately 30 kDa. The polypeptides form typical collagen triple helices that are cross-linked through tyrosine-tyrosine bonds. The triple helices are stabilized within the cuticle by interchain disulfide bonds (Betschart and Wyss 1990); Fetterer and Rhodes 1993). In cuticular collagens, the cross-linking amino acid isotrityrosine is present in significant amounts and dityrosine is present in lesser amounts. Nematode cuticular collagens differ from vertebrate collagens. Nematode collagens have non-repeating regions interspersed among the typical collagen like repeat units while vertebrate collagens do not (Shamansky et al. 1989; Kingston and Pettitt 1990).

Insoluble cuticular collagen is found primarily in the external cortical layer and the epicuticle. This protein was first identified in A. suum and termed cuticulin (Fujimoto 1975). Although cuticulin is similar to collagen in proline and glycine content, it is insensitive to digestion by bacterial collagenase (Fujimoto and Kanaya 1973). Physical characterization of cuticulin is difficult due to its insolubility. In the nematode cuticle, tyrosine residues of both collagens and cuticulin are post translationally modified by the formation of ditryrosine and isotrityrosine cross-links. Ditryrosine and isotritrysoine cross-links are presumably formed by the action of peroxidases on adjacent tyrosine residues (Amado et al. 1984; Fry 1987). Cuticulin has significant amounts of dityrosine.

The isolation and characterization of a serratia metalloprotease (SMP) isoform isolated from Serratia marcescens strain NRRL B-23112 has been previously reported (Salamone and Wodzinski, 1997); however, now Applicants have isolated and sequenced the polynucleotide which encodes this polypeptide. Surprisingly, this protein has been found to be deleterious to several species of economically important nematodes.

Thus, there is a need to have to have a more effective and safe means to control parasitic nematodes without causing health and environmental problems.

### BRIEF SUMMARY OF THE INVENTION

The present invention provides polynucleotides, related polypeptides and all conservatively modified variants of a newly discovered SMP, as well as a newly discovered serratia metalloprotease inhibitor (SmaPI). The full-length nucleotide and amino acid sequences of this newly discovered SMP comprise, respectively, the sequences found in SEQ ID NO: 1 and SEQ ID NO:2. The full-length nucleotide and amino acid sequence of the newly discovered SmaPI comprise, respectively, the sequences found in SEQ ID NO:3 and SEQ ID NO:4.

In a first embodiment, there is provided an isolated nucleic acid molecule comprising a polynucleotide selected from the group consisting of: (a) a polynucleotide having 70% sequence identity to SEQ ID NO: 1; (b) a polynucleotide having 80% sequence identity to SEQ ID NO: 1; (c) a polynucleotide having 90% sequence identity to SEQ ID NO:1; (d) a polynucleotide having 95% sequence identity to SEQ ID NO:1; (e) a polynucleotide having the sequence of SEQ ID NO:1; (f) a polynucleotide encoding a polypeptide having 70% sequence identity to SEQ ID NO:2; (g) a polynucleotide encoding a polypeptide having 80% sequence identity to SEQ ID NO:2; (h) a polynucleotide encoding a polypeptide having 90% sequence identity to SEQ ID NO:2; (i) a polynucleotide encoding a polypeptide having 95% sequence identity to SEQ ID NO:2; (j) a polynucleotide encoding the polypeptide of SEQ ID NO:2; (k) a polynucleotide that hybridizes under conditions of high stringency to the polynucleotide of SEQ ID NO:1; and (l) a polynucleotide complementary to a polynucleotide of (a) through (k).

In another embodiment, there is provided an isolated nucleic acid molecule comprising a polynucleotide selected from the group consisting of: (a) a polynucleotide having 70% sequence identity to SEQ ID NO: 3; (b) a polynucleotide having 80% sequence identity to SEQ ID NO: 3; (c) a polynucleotide having 90% sequence identity to SEQ ID NO:3; (d) a polynucleotide having 95% sequence identity to SEQ ID NO:3; (e) a polynucleotide having the sequence of SEQ ID NO:3; (f) a polynucleotide encoding a polypeptide having 70% sequence identity to SEQ ID NO:4; (g) a polynucleotide encoding a polypeptide having 80% sequence identity to SEQ ID NO:4; (h) a polynucleotide encoding a polypeptide having 90% sequence identity to SEQ ID NO:4; (i) a polynucleotide encoding a polypeptide having 95% sequence identity to SEQ ID NO:4; (j) a polynucleotide encoding the polypeptide of SEQ ID NO:4; (k) a polynucleotide that hybridizes under conditions of high stringency to the polynucleotide of SEQ ID NO:3; and (l) a polynucleotide complementary to a polynucleotide of (a) through (k).

In another embodiment, there is provided a recombinant expression cassette comprising one or both of the described nucleic acid molecules. Additionally, the present invention provides a vector containing the recombinant expression cassette comprising one or both of the described nucleic acid molecules. Further, the vector containing the recombinant expression cassette can facilitate the transcription and translated of the nucleic acid molecule(s) in a host cell. A number of host cells could be used, such as, but not limited to, microbial, mammalian, plant, or insect.

In yet another embodiment, there isprovided a transgenic plant or plant cells, containing one or both of the described nucleic acid molecules. Preferred plants include any plant that may be susceptible to nematode infection including, but not limited to, maize, soybean, sunflower, sorghum, canola, wheat, alfalfa, cotton, rice, barley, tomato, banana, citrus, avocado, and millet. In yet another embodiment are the seeds from the transgenic plant.

In yet another embodiment, there is provided an isolated polypeptide comprising a polypeptide selected from the group consisting of: (a) a polypeptide having an amino acid sequence 70% identical to the amino acid sequence of SEQ ID NO:2; (b) a polypeptide having an amino acid sequence 80% identical to the amino acid sequence of SEQ ID NO:2; (c) a polypeptide having an amino acid sequence 90% identical to the amino acid sequence of SEQ ID NO:2; (d) a polypeptide having an amino acid sequence 95% identical to the amino acid sequence of SEQ ID NO:2; (e) a polypeptide having the amino acid sequence of SEQ ID NO:2; and (f) a polypeptide which is encoded by the polynucleotide of SEQ ID NO:1.

In yet another embodiment, there is provided a method for making a SMP comprising expressing the polynucleotide of SEQ ID NO:1, or variants thereof, in a recombinantly engineered cell, wherein the polynucleotide is operably linked to a promoter such that the polypeptide is produced; and obtaining the resultant protein. In yet another embodiment, there is provided a method for controlling a pest comprising administering to the pest a polypeptide having the amino acid sequence of SEQ ID NO: 2. In one aspect, the pest is a nematode belonging to the genera selected from the group consisting of Ascaris and Radopholus.

In yet another embodiment, there is provided an isolated polypeptide comprising a polypeptide selected from the group consisting of: (a) a polypeptide having an amino acid sequence 70% identical to the amino acid sequence of SEQ ID NO:4; (b) a polypeptide having an amino acid sequence 80% identical to the amino acid sequence of SEQ ID NO:4; (c) a polypeptide having an amino acid sequence 90% identical to the amino acid sequence of SEQ ID NO:4; (d) a polypeptide having an amino acid sequence 95% identical to the amino acid sequence of SEQ ID NO:4; (e) a polypeptide having the amino acid sequence of SEQ ID NO:4; and (f) a polypeptide which is encoded by the polynucleotide of SEQ ID NO:3.

In yet another embodiment, there is provided a method for making a SmaPI comprising expressing the polynucleotide of SEQ ID NO:3, or variants thereof, in a recombinantly engineered cell, wherein the polynucleotide is operably linked to a promoter such that the polypeptide is produced; and obtaining the resultant protein.

In yet another embodiment, there is provided a method for conferring nematode resistance to a plant comprising stably transforming the plant with an expression construct comprising the polynucleotide of SEQ ID NO:1 under the control of a promoter operably linked thereto. The construct can additionally comprise the polynucleotide of SEQ ID NO:3, which can be under the control of the same promoter.

### BRIEF DESCRIPTION OF THE DRAWINGS

FIG. 1 provides the nucleotide and amino acid sequence of the newly discovered SMP (SEQ ID NOS:1 and 2, respectively). This SMP was isolated from S. marcescens NRRL B-23112.

FIG. 2 provides the nucleotide sequence and amino acid sequence of the newly discovered SmaPI (SEQ ID NOS:3 and 4, respectively). This SmaPI was isolated from S. marcescens ATCC 27117.

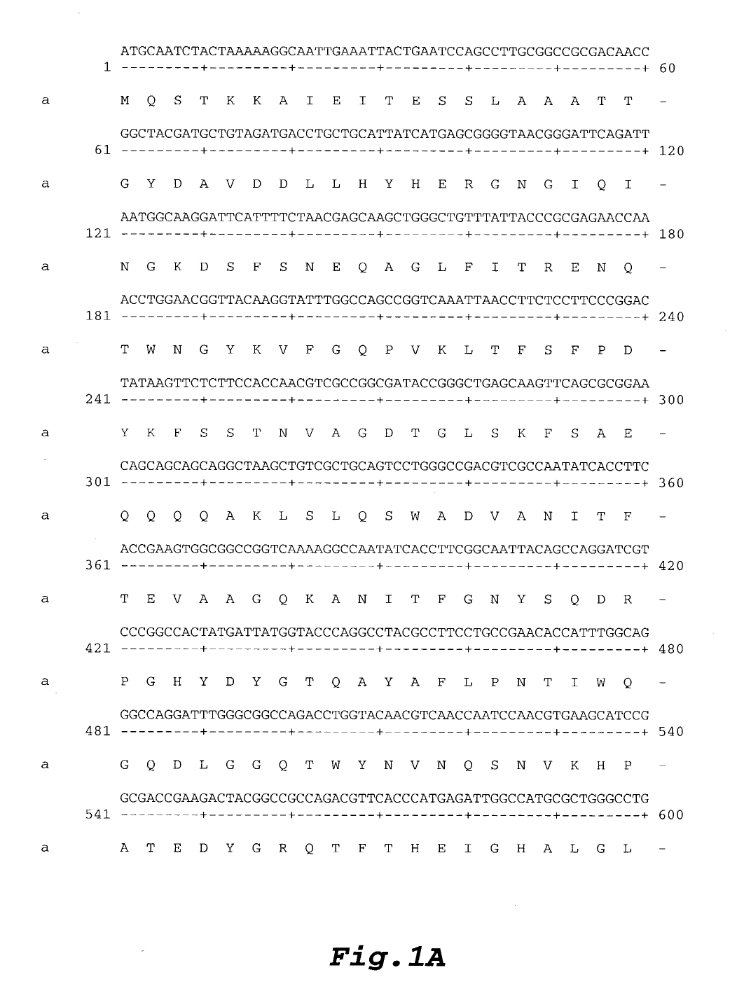

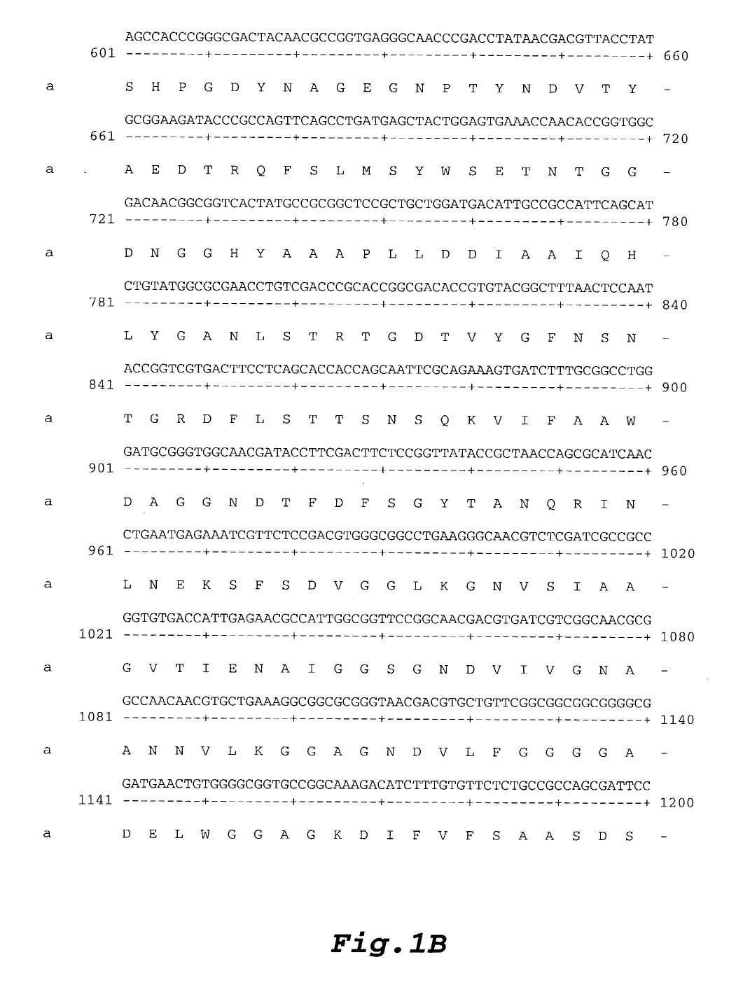

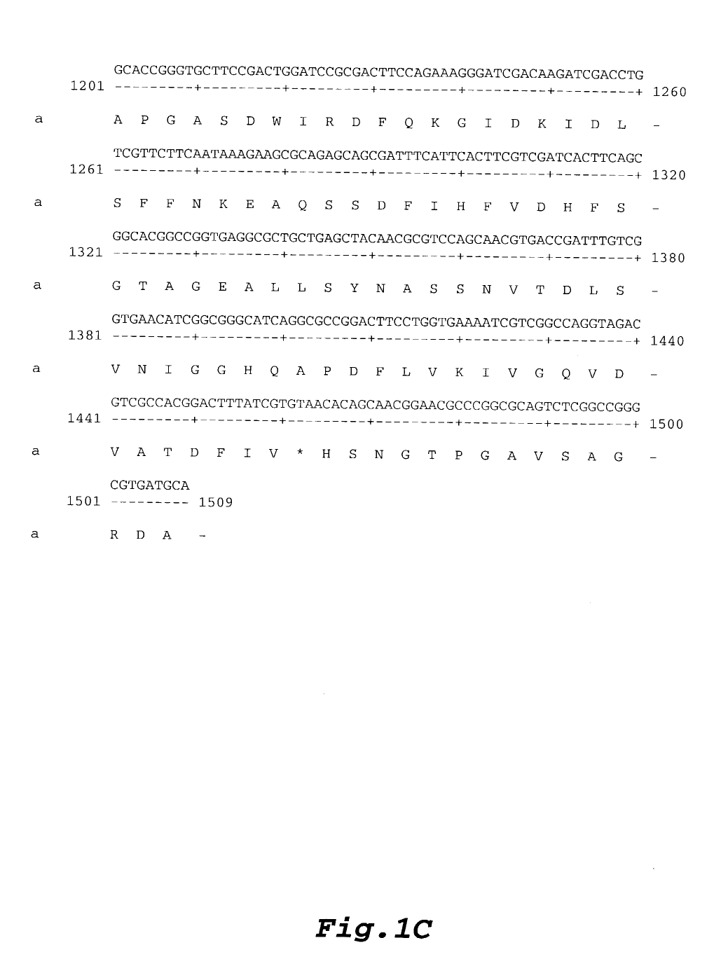

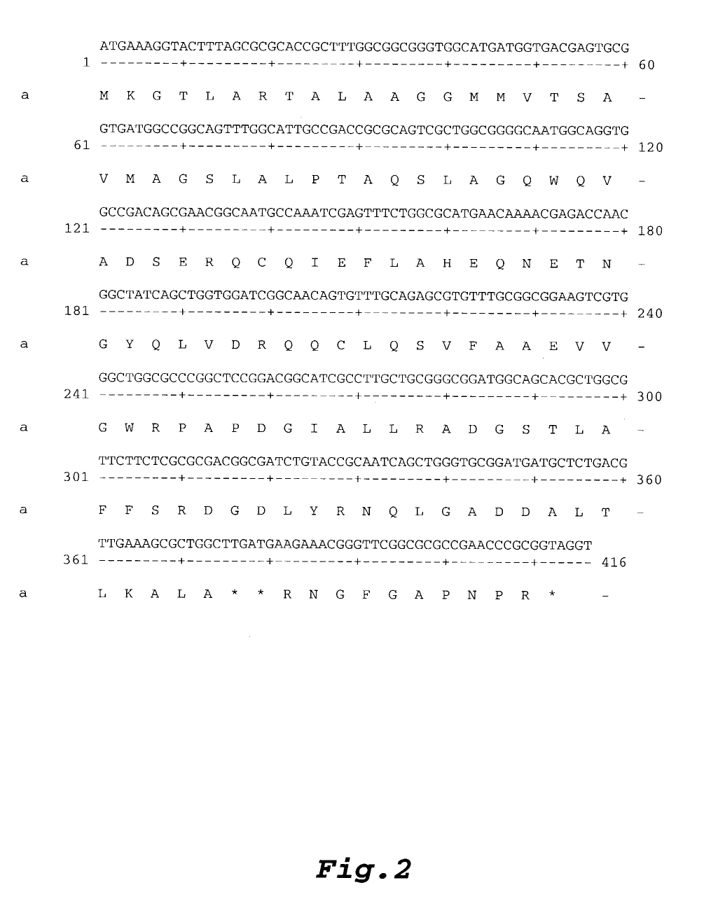

### DETAILED DESCRIPTION OF THE INVENTION

An embodiment of the present invention relates to a polynucleotide encoding a SMP, sometimes referred to herein as “SMP #1,” which has selective nematolytic activity and which has sequence homology with another SMP, serratiopeptidase, sometimes referred to herein as “SMP #2” (Sigma; Genbank accession no. X55521.

The SMP #1 is useful for the control of pests having collagenous cuticles. This SMP is further useful as a nematicide for controlling plant parasitic, whether free-living or burrowing, or endoparasitic nematodes. This SMP is particularly useful for controlling nematodes of the genera Ascaris or Radopholus.

One critical characteristic of SMP #1 described herein is its selective pesticidal activity. It has surprisingly been discovered that when SMP #1 comes into contact with A. suum or R. similis, it lyses the nematode. The cuticle and body wall are lysed and the internal viscera expelled within 30 hours. Serratiopeptidase (SMN #2), however, does not lyse or cause significant mortality of any of the nematodes.

A direct comparison of serratiopeptidase with SMP #1 indicated that the isoelectric point, peptide mapping, and lytic activity against live A. suum and R. similis, and A. suum cuticular components in vitro differed. Peptide mapping of SMP #1 and serratiopeptidase after digestion with trypsin and Glu-C revealed distinct differences. The digestion patterns indicate a difference in either amino acid stoichiometry or distribution.

SMP #1 is inhibited by EDTA (9μg/ml), and not inhibited by antipain dihydrochloride (120 μg/ml), aprotinin (4 μg/ml), bestatin (80 μg/ml), chymostatin (50 μg/ml), E-64 (20 μg/ml), leupeptin (4 μg/ml), Pefabloc SC (2000 μ/ml), pepstatin (4 μg/ml), phosphoramidon (660 μ/ml), or phenylmethylsulfonyl fluoride (400 μ/ml). It however retains full activity in the presence of SDS (1% w/v), Tween-20 (1% w/v), Triton X-100 (1% w/v), ethanol (5% v/v), and 2-mercaptoethanol (0.5% v/v).

Another embodiment of the present invention relates to a novel serratia metalloprotease inhibitor (SmaPI), sometimes referred to herein as “SmaPI #1,” which inhibits the activity of the presently discovered SMP, as well as other previously disclosed SMPs. This newly discovered SmaPI (SEQ ID NO:3) shares sequence homology with previously described SmaPIs (i.e. Genbank accession no. X55521 and L09107). The SmaPI polynucleotide of the present invention is useful, for example, when it is co-expressed expressed in a plant cell with a SMP-encoding polynucleotide in such a manner that the SMP does not become toxic to the cell.

The practice of the present invention will employ, unless otherwise indicated, conventional techniques of botany, microbiology, tissue culture, molecular biology, chemistry, biochemistry and recombinant DNA technology, which are within the skill of the art. Such-techniques are explained fully in the literature. See, e.g., J. H. Langenheim and K. V. Thimann, Botany: Plant Biology and Its Relation to Human Affairs (1982) John Wiley; Cell Culture and Somatic Cell Genetics of Plants, Vol. 1 (I. K. Vasil, ed. 1984); R. V. Stanier, J. L. Ingraham, M. L. Wheelis, and P. R. Painter, The Microbial World, (1986) 5th Ed., Prentice-Hall; O. D. Dhringra and J. B. Sinclair, Basic Plant Pathology Methods, (1985) CRC Press; Maniatis, Fritsch & Sambrook, Molecular Cloning: A Laboratory Manual (1982); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); and the series Methods in Enzymology (S. Colowick and N. Kaplan, eds., Academic Press, Inc.).

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed, and are intended to be defined as indicated below and herein.

As used herein, the term “SMP” is intended to refer to a metalloprotease isolated from Serratia spp. The term “SmaPI” is intended to refer to a metalloprotease inhibitor isolated from Serratia spp.

By “microbe” is meant any microorganism (including both eukaryotic and prokaryotic microorganisms), such as fungi, yeast, bacteria, actinomycetes, algae and protozoa, as well as other unicellular structures.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Proc. Natl. Acad. Sci. (USA), 82: 2306-2309 (1985)), or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray et al. Nucl. Acids Res. 17: 477-498 (1989) and herein incorporated by reference. Thus, the maize preferred codon for a particular amino acid might be derived from known gene sequences from maize. Maize codon usage for 28 genes from maize plants is listed in Table 4 of Murray et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” or “recombinantly engineered cell” is meant a cell, which contains a vector and supports the replication and/or expression of the expression vector. Host cells may be prokaryotic cells such as E. coli, or eukaryotic cells such as yeast, Pichia, insect, plant, amphibian, or mammalian cells. Preferably, host cells are monocotyledonous or dicotyledonous plant cells, including but not limited to maize, sorghum, sunflower, soybean, wheat, alfalfa, rice, cotton, canola, barley, millet, and tomato, avacodo, banana or citrus. Preferred host cells are a tomato, citrus, soybean, avocado, or banana. A particularly preferred host cell is banana.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The terms “polypeptide”,“peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen, and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, Citrus, Persea, Musa, and Triticum. A particularly preferred plant is Musa.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibres, xylem vessels, tracheids, or sclerenchyma. Such promoters are referred to as “tissue preferred”. A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated, and inducible promoters constitute the class of “non-constitutive” promoters. A “constitutive” promoter is a promoter, which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994 Supplement).

The microorganism useful according to the subject invention has been deposited in the permanent collection of the Agricultural Research Service Patent Culture Collection (NRRL), Northern Regional Research Center, 1815 North University Street, Peoria, Ill. 61604, USA. The culture repository number of the deposited strain is as follows:

| Culture | Repository No. | Deposit Date |
|---|---|---|
| S. marcescens | NRRL-B-23112 | Dec. 9, 1996 |

This isolate has been deposited under condition that assure that access to the culture will be available during the pendency of this patent application to one determined by the Commissioner of Patents and Trademarks to be entitled thereto under 37 CFR 1.14 and 35 USC 122. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

Following is a table which provides characteristics of the polypeptide produced by a soil isolate of S. marcescens NRRL B-23112.

TABLE 1
Description of S. marcescens Strain NRRL
B-23112 toxic to nematodes

| | Crystal | Optimum | Isoelectric | Approx. |
|---|---|---|---|---|
| Culture | Structure | pH | point | M.W. (Da) |
| S. marcescens | R-Factor | 10 | 6.1 | 50,900 |

The novel polypeptide and polynucleotide sequences provided herein are defined according to several parameters. The polypeptides and polynucleotides of the subject invention can be defined by their amino acid and nucleotide sequences. The nucleotide sequences of the subject polynucleotides are provided as SEQ ID NO: 1 and SEQ ID NO:3. The amino acid sequences of the subject polypeptides are provided as SEQ ID NO: 2 and SEQ ID NO:4.

The sequence of the molecule can be defined herein in terms of homology to the exemplified sequence as well as in terms of the ability to hybridize with, or be amplified by, certain exemplified probes and primers. The polypeptides provided herein can also be identified based on their immunoreactivity with certain antibodies.

The polypeptides and polynucleotides of the subject invention can be identified and obtained by using oligonucleotide probes, for example, these probes are detectable nucleotide sequences. The probes (and the polynucleotides of the subject invention) may be DNA, RNA, or PNA (peptide nucleic acid). These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (%GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

Detection of the probe provides a means for determining in a known manner whether hybridization has occurred. Such a probe analysis provides a rapid method for identifying the genes of the subject invention. The nucleotide segment which is used as probes according to the invention can be synthesized using a DNA synthesizer and standard procedures. This nucleotide sequence can also be used as PCR primers to amplify genes of the subject invention.

Polymerase Chain Reaction (PCR) is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. et al. (1985) “Enzymatic Amplification of β-Globin Genomic Sequences and Restriction Site Analysis for Diagnosis of Sickle Cell Anemia,” Science 230:1350-1354). PCR is based on the enzymatic amplification of a DNA fragment of interest that is flanked by two oligonucleotide primers that hybridize to opposite strands of the target sequence. By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

The primers are oriented with the 3′ ends pointing towards each other. Repeated cycles of heat denaturation of the template, annealing of the primers to their complementary sequences, and extension of the annealed primers with a DNA polymerase results in the amplification of the segment defined by the 5′ ends of the PCR primers. Since the extension product of each primer can serve as a template for the other primer, each cycle essentially doubles the amount of DNA fragment produced in the previous cycle. This results in the exponential accumulation of the specific target fragment, up to several million-fold in a few hours. By using a thermostable DNA polymerase such as Taq polymerase, which is isolated from the thermophilic bacterium Thermus aquaticus, the amplification process can be completely automated. Other enzymes which can be used are known to those skilled in the art.

PCR primers can be designed from the DNA sequences of the subject invention. In performing PCR amplification, a certain degree of mismatch can be tolerated between primer and template. Therefore, mutations, deletions and insertions (especially additions of nucleotides to the 5′ end) of the exemplified sequences fall within the scope of the subject invention. These PCR primers can be used to amplify genes of interest from a sample. Thus, this is another method by which polynucleotide sequences encoding the subject peptides can be identified and characterized.

The various methods employed in the preparation of plasmids comprising the pesticidal polypeptide encoding polynucleotides of the present invention, and transformation of host organisms are well known in the art and are described, for example, in U.S. Pat. Nos. 5,011,909 and 5,130,253. These patents are incorporated herein by reference. These procedures are also described in Maniatis et al. (1982) Molecular Cloning: A Laboratory Manual, Cold Spring Harbor Laboratory, New York. Thus, it is within the skill of those in the genetic engineering art to extract DNA from microbial cells, perform restriction enzyme digestions, electrophorese DNA fragments, tail and anneal plasmid and insert DNA, ligate DNA, transform cells, e.g., E. coli or plant cells, prepare plasmid DNA, electrophorese proteins, and sequence DNA.

The polynucleotides and polypeptides useful according to the subject invention includes not only the specifically exemplified full-length sequences, but also portions (including internal deletions compared to the full-length proteins), fragments (including terminal deletions compared to the full-length protein) of these sequences, variants, mutants, chimeric, and fusion proteins, including proteins having substituted amino acids, which retain the characteristic activity of the proteins specifically exemplified herein.

As used herein, the terms “variants” or “variations” of genes refer to nucleotide sequences which encode the same polypeptides or which encode equivalent polypeptides.

As used herein, the term “equivalent polypeptides” refers to polypeptides having the same or essentially the same biological activity as the claimed polypeptide. Equivalent polypeptides and/or polynucleotides encoding these equivalent polypeptides can be derived from the relevant S. marcescens isolate and/or a nucleic acid library.

There are a number of methods for obtaining polypeptides of the instant invention. For example, antibodies to the polypeptides disclosed and claimed herein can be used to identify and isolate other polypeptides from a mixture of proteins. Specifically, antibodies may be raised to the portions of the polypeptide which is most constant and most distinct from other S. marcescens polypeptides. These antibodies can then be used to specifically identify equivalent polypeptides with the characteristic activity by immunoprecipitation, enzyme linked immunosorbent assay (ELISA), or western blotting. Antibodies to the polypeptides or to equivalent polypeptides, or fragments of these polypeptides, can readily be prepared using standard procedures in this art. The polynucleotides which encode the polypeptides can then be obtained from the microorganism.

Also, because of the redundancy of the genetic code, a variety of different DNA sequences can encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine, one exception is Micrococcus rubens, for which GTG is the methionine codon (Ishizuka, et al., J. Gen'l Microbiol, 139:425-432 (1993)) can be modified to yield a functionally identical molecule. Accordingly, each silent variation of a nucleic acid, which encodes a polypeptide of the present invention, is implicit in each described polypeptide sequence and incorporated herein by reference.

It is well within the skill of a person trained in the art to create these alternative DNA sequences encoding the same, or essentially the same polypeptide. These variant DNA sequences are within the scope of the subject invention. As used herein, reference to “essentially the same” sequence refers to sequences which have amino acid substitutions, deletions, additions, or insertions which do not materially affect activity. Fragments retaining activity are also included in this definition.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90%, preferably 60-90% of the native protein for it's native substrate.

The amino acid homology will be highest in critical regions of the polypeptides which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Table 2 provides a listing of examples of amino acids belonging to each class.

TABLE 2

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glw |
| Basic | Lys, Arg, His |

In some instances, non-conservative substitutions can also be made. The critical factor is that these substitutions must not significantly detract from the biological activity of the polypeptides.

Synthetic genes which are functionally equivalent to the polynucleotides of the subject invention can also be used to transform hosts. Methods for the production of synthetic genes can be found in, for example, U.S. Pat. No. 5,380,831. See also, Creighton (1984) Proteins W. H. Freeman and Company.

Equivalent polypeptides will have amino acid homology with exemplified polypeptides. The amino acid identity will typically be greater than 60%, preferably be greater than 70%, more preferably greater than 80%, more preferably greater than 90%, and can be greater than 95%.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (Best Fit) of Smith and Waterman, Adv. Appl. Math may conduct optimal alignment of sequences for comparison. 2: 482 (1981); by the homology alignment algorithm (GAP) of Needleman and Wunsch, J. Mol. Biol. 48: 443 (1970); by the search for similarity method (Tfasta and Fasta) of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The preferred program to use for optimal global alignment of multiple sequences is PileUp (Feng and Doolittle, Journal of Molecular Evolution, 25:351-360 (1987) which is similar to the method described by Higgins and Sharp, CABIOS, 5:151-153 (1989) and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50, or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. USA 89:10915).

Unless otherwise stated, sequence identity/similarity values provided herein refer to the value obtained using the BLAST 2.0 suite of programs using default parameters. Altschul et al., Nucleic Acids Res. 25:3389-3402 (1997).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 40-100%, preferably at least 55%, preferably at least 60%, more preferably at least 70%, 80%, 90%, and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, J. Mol. Biol. 48: 443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′ SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSlox, and lambda MOSElox. Optional vectors for the present invention, include but are not limited to, lambda ZAP II, and pGEX. For a description of various nucleic acids see, for example, Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang et al., Meth. Enzymol. 68: 90-99 (1979); the phosphodiester method of Brown et al., Meth. Enzymol. 68: 109-151 (1979); the diethylphosphoramidite method of Beaucage et al., Tetra. Lett. 22: 1859-1862 (1981); the solid phase phosphoramidite triester method described by Beaucage and Caruthers, Tetra. Letts. 22(20): 1859-1862 (1981), e.g., using an automated synthesizer, e.g., as described in Needham-VanDevanter et al., Nucleic Acids Res., 12: 6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, Nucleic Acids Res. 15:8125 (1987)) and the 5<G>7 methyl GpppG RNA cap structure (Drummond et al., Nucleic Acids Res. 13:7375 (1985)). Negative elements include stable intramolecular 5′ UTR stem-loop structures (Muesing et al., Cell 48:691 (1987)) and AUG sequences or short open reading frames preceded by an appropriate AUG in the 5′ UTR (Kozak, supra, Rao et al., Mol. and Cell. Biol. 8:284 (1988)). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12: 387-395 (1984)) or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50, or 100

### Sequence Shuffling

The present invention contemplates sequence shuffling using polynucleotides of the present invention, and compositions resulting therefrom. Sequence shuffling is described in PCT publication No. 96/19256. See also, Zhang, J.- H., et al. Proc. Natl. Acad. Sci. USA 94:4504-4509 (1997) and Zhao, et al., Nature Biotech 16:258-261 (1998). Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a substrate binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present invention further provides recombinant expression cassettes comprising nucleic acids of the present invention. A nucleic acid sequence coding for the desired polynucleotide of the present invention, for example a cDNA or a genomic sequence encoding a polypeptide long enough to code for an active protein of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell et al., (1985), Nature, 313:810-812, rice actin (McElroy et al., (1990), Plant Cell, 163-171); ubiquitin (Christensen et al., (1992), Plant Mol. Biol. 12:619-632; and Christensen, et al., (1992), Plant Mol. Biol. 18:675-689); pEMU (Last, et al., (1991), Theor. Appl. Genet. 81:581-588); MAS (Velten et al., (1984), EMBO J. 3:2723-2730); and maize H3 histone (Lepetit et al., (1992), Mol. Gen. Genet. 231:276-285; and Atanassvoa et al., (1992), Plant Journal 2(3):291-300), the Rsyn7 as described in published PCT Application WO 97/44756, ALS promoter, as described in published PCT Application WO 96/30530, and other transcription initiation regions from various plant genes known to those of skill. For the present invention ubiquitin is the preferred promoter for expression in monocot plants.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adhl promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes, or alternatively from another plant gene, or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan et al., (1983), Nucl. Acids Res. 12:369-385); the potato proteinase inhibitor II (PINII) gene (Keil, et al., (1986), Nucl. Acids Res. 14:5641-5650; and An et al., (1989), Plant Cell 1:115-122); and the CaMV 19S gene (Mogen et al., (1990), Plant Cell 2:1261-1272).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8: 4395-4405 (1988); Callis et al., Genes Dev. 1: 1183-1200 (1987). Such intron enhancement of gene expression is typically greatest when placed near the 5′ end of the transcription unit. Use of maize introns Adh 1-S intron 1, 2, and 6, the Bronze-1 intron are known in the art. See generally, The Maize Handbook, Chapter 116, Freeling and Walbot, Eds., Springer, New York (1994).

Plant signal sequences, including, but not limited to, signal-peptide encoding DNA/RNA sequences which target proteins to the extracellular matrix of the plant cell (Dratewka-Kos, et al., (1989), J. Biol. Chem. 264:4896-4900), the Nicotiana plumbaginifolia extension gene (DeLoose, et al., (1991), Gene 99:95-100), signal peptides which target proteins to the vacuole like the sweet potato sporarmin gene (Matsuka, et al., (1991), PNAS 88:834) and the barley lectin gene (Wilkins, et al., (1990), Plant Cell, 2:301-313), signal peptides which cause proteins to be secreted such as that of PRIb (Lind, et al., (1992), Plant Mol. Biol. 18:47-53), or the barley alpha amylase (BAA) (Rahmatullah, et al., Plant Mol. Biol. 12:119 (1989)) and hereby incorporated by reference), or from the present invention the signal peptide from the ESP1 or BEST1 gene, or signal peptides which target proteins to the plastids such as that of rapeseed enoyl-Acp reductase (Verwaert, et al., (1994), Plant Mol. Biol. 26:189-202) are useful in the invention. For expression in a plant, the nucleotide sequence is fused to a plant signal sequence.

The vector comprising the sequences from a polynucleotide of the present invention will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta, and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers et al., Meth. In Enzymol., 153:253-277 (1987). These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl et al., Gene, 61:1-11 (1987) and Berger et al., Proc. Natl. Acad. Sci. U.S.A., 86:8402-8406 (1989). Another useful vector herein is plasmid pBI101.2 that is available from CLONTECH Laboratories, Inc. (Palo Alto, Calif.).

### Expression of Proteins in Host Cells

Using the nucleic acids of the present invention, one may express proteins of the present invention within a recombinantly engineered cell such as microbial, yeast, insect, mammalian, or preferably plant cells, or cells derived therefrom. Additionally, one may cause the proteins to be secreted from such cells The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding proteins of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation, and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level”, or about 1/10 transcripts to about 1/100 transcripts to about 1/1,000 transcripts.

One of skill would recognize that modifications could be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

A. Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang et al., Nature 198:1056 (1977)), the tryptophan (trp) promoter system (Goeddel et al., Nucleic Acids Res. 8:4057 (1980)) and the lambda derived P L promoter and N-gene ribosome binding site (Shimatake et al., Nature 292:128 (1981)). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., Gene 22: 229-235 (1983); Mosbach, et al., Nature 302: 543-545 (1983)). The pGEX-4T-1 plasmid vector from Pharmacia is the preferred E. coli expression vector for the present invention.

B. Expression in Eukaryotes

A variety of eukaryotic expression systems such as yeast, insect cell lines, plant and mammalian cells, are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, F., et al., Methods in Yeast Genetics, Cold Spring Harbor Laboratory (1982) is a well recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Mammalian cell systems often will be in the form of monolayers of cells although mammalian cell suspensions may also be used. A number of suitable host cell lines capable of expressing intact proteins have been developed in the art, and include the HBEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen et al., Immunol. Rev. 89: 49 (1986)), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7th edition, 1992).

Appropriate vectors for expressing proteins of the present invention in insect cells are usually derived from the SF9 baculovirus. Suitable insect cell lines include mosquito larvae, silkworm, armyworm, moth, and Drosophila cell lines such as a Schneider cell line (See Schneider, J. Embryol. Exp. Morphol. 27: 353-365 (1987).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al., J. Virol. 45: 773-781 (1983)). Additionally, gene sequences to control replication in the host cell may be incorporated into the vector such as those found in bovine papilloma virus type-vectors. Saveria-Campo, M., Bovine Papilloma Virus DNA a Eukaryotic Cloning Vector in DNA Cloning Vol. II a Practical Approach, D. M. Glover, Ed., IRL Press, Arlington, Va. pp. 213-238 (1985).

In addition, the SMP #1- and/or SmaPI #1-encoding polynucleotides placed in the appropriate plant expression vector can be used to transform plant cells. The resultant proteins can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert one or more instantly disclosed polynucleotides into a plant host, including biological and physical plant transformation protocols. See, for example, Miki et al., (1993), “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch, et al., (1985), Science 227:1229-31), electroporation, micro-injection, and biolistic bombardment.

Expression cassettes and vectors and in vitro culture methods for plant cell or tissue transformation and regeneration of plants are known and available in the art. See, for example, Gruber, et al., (1993), “Vectors for Plant Transformation” In: Methods in Plant Molecular Biology and Biotechnology, Glick and Thompson, eds. CRC Press, Inc., Boca Raton, pages 89-119.

### Agrobacterium-mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, for example, Kado, (1991), Crit. Rev. Plant Sci. 10:1. Descriptions of the Agrobacterium vector systems and methods for Agrobacterium-mediated gene transfer are provided in Gruber et al., supra; Miki, et al., supra; and Moloney et al., (1989), Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey, P. N., and Chua, N. H. (1989) Science 244: 174-181. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. application Ser. No. 913,914, filed Oct. 1, 1986, as referenced in U.S. Patent No. 5,262,306, issued Nov. 16, 1993 to Robeson, et al. ; and Simpson, R. B., et al. (1986) Plant Mol. Biol. 6: 403-415 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to Radopholus infection. Several other transgenic plants are also contemplated by the present invention including but not limited to soybean, corn, sorghum, alfalfa, rice, clover, cabbage, banana, coffee, celery, tobacco, cowpea, cotton, melon, pepper, avacodo, tomato, and citrus. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g. certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application Publication Number 604 662 A1 to Hici et al. discloses a method for transforming monocots using Agrobacterium. Saito et al. discloses a method for transforming monocots with Agrobacterium using the scutellum of immature embryos (European Application 672 752 A1). Ishida et al. discusses a method for transforming maize by exposing immature embryos to A. tumefaciens (Ishida et al., Nature Biotechnology, 1996, 14:745-750).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the instantly disclosed polynucleotides, can be used as a source of plant tissue to regenerate nematode-resistant transgenic plants, either via somatic embryogenesis or organogenesis. Examples of such methods for regenerating plant tissue are disclosed in Shahin, E. A. (1985) Theor. Appl. Genet. 69:235-240; U.S. Pat. No. 4,658,082; Simpson, R. B., et al. (1986) Plant Mol. Biol. 6: 403-415; and U.S. patent applications Ser. Nos. 913,913 and 913,914, both filed Oct. 1, 1986, as referenced in U.S. Patent No. 5,262,306, issued Nov. 16, 1993 to Robeson, et al. ; the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei et al., (1994), The Plant Journal 6:271-282). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford et al., (1987), Part. Sci. Technol. 5:27; Sanford, 1988, Trends Biotech 6:299; Sanford, (1990), Physiol. Plant 79:206; Klein et al., (1992), Biotechnology 10:268).

Another method for physical delivery of DNA to plants is sonication of target cells as described in Zang et al., (1991), BioTechnology 9:996. Alternatively, liposome or spheroplast fusions have been used to introduce expression vectors into plants. See, for example, Deshayes et al., (1985), EMBO J. 4:2731; and Christou et al., (1987), PNAS USA 84:3962. Direct uptake of DNA into protoplasts using CaCl₂ precipitation, polyvinyl alcohol, or poly-L-ornithine has also been reported. See, for example, Hain et al., (1985), Mol. Gen. Genet. 199:161; and Draper et al., (1982), Plant Cell Physiol. 23:451.

Electroporation of protoplasts and whole cells and tissues has also been described. See, for example, Donn et al., (1990), In: Abstracts of the VIIth Int'l. Congress on Plant Cell and Tissue Culture IAPTC, A2-38, page 53; D'Halluin et al., (1992), Plant Cell 4:1495-1505; and Spencer et al., Plant Mol. Biol. 24:51-61 (1994).

### Recombinant Hosts

The polynucleotide(s) of the subject invention can be introduced into a wide variety of microbial or plant hosts. Expression of the polynucleotide(s) results, directly or indirectly, in the intracellular production or secretion and maintenance of the protein. The target pest can contact SMP #1 by contacting plant tissue containing SMP #1, which is toxic to the pest. The result is control of the pest. Alternatively, suitable microbial hosts, e.g., Pseudomonas, can be applied to the situs of the pest, where some of which can proliferate, and are contacted by the target pests. The microbe hosting the SMP #1-encoding polynucleotide can be treated under conditions that prolong the activity of SMP #1 and stabilize the cell. The treated cell, which retains the toxic activity, then can be applied to the environment of the target pest.

Where the polynucleotide(s) of the subject invention is introduced via a suitable vector into a microbial host, and said host is applied to the environment in a living state, certain host microbes should be used. Microorganism hosts are selected which are known to occupy the “phytosphere” (phylloplane, phyllosphere, rhizosphere, and/or rhizoplane) of one or more crops of interest. These microorganisms are selected so as to be capable of successfully competing in the particular environment (crop and other insect habitats) with the wild-type microorganisms, provide for stable maintenance and expression of the polynucleotide and, desirably, provide for improved protection of the polypeptide from environmental degradation and inactivation.

A large number of microorganisms are known to inhabit the rhizosphere (the soil surrounding plant roots). These microorganisms include bacteria, algae, and fungi. Of particular interest are microorganisms, such as bacteria, e.g., genera Pseudomonas, Erwinia, Serratia, Klebsiella, Xanthomonas, Streptomyces, Rhizobium, Rhodopseudomonas, Methylophilius, Agrobacterium, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacterium tumefaciens, Rhodopseudomonas spheroides, Xanthomonas campestris, Rhizobium melioti, Alcaligenes entrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A wide variety of ways are available for introducing a polynucleotide of the subject invention into the target host under conditions which allow for stable maintenance and expression of the polynucleotide. These methods are well known to those skilled in the art and are described, for example, in U.S. Pat. No 5,135,867, which is incorporated herein by reference.

### Treatment of Cells

S. marcescens, or recombinant cells expressing the presently disclosed polynucleotide(s), can be treated to increase the activity of the polypeptide encoded by the polynucleotide and stabilize the cell. The delivery vector that is formed comprises the polypeptide within a cellular structure that has been transformed and will deliver the polypeptide when the host organism is applied to the environment of the target pest. Suitable host cells may include either prokaryotes or eukaryotes, normally being limited to those cells which do not produce substances toxic to higher organisms, such as mammals. However, organisms which produce substances toxic to higher organisms could be used, where the toxic substances are unstable or the level of application sufficiently low as to avoid any possibility of toxicity to a mammalian host. As hosts, of particular interest will be the prokaryotes and the lower eukaryotes, such as fungi.

The cell will usually be intact and be substantially in the proliferative form when treated, rather than in a spore form, although in some instances spores may be employed.

Treatment of the microbial cell, e.g., a microbe containing the instantly disclosed polynucleotide(s), can be by chemical or physical means, or by a combination of chemical and/or physical means, so long as the technique does not deleteriously affect the properties of the polypeptide(s), nor diminish the cellular capability of protecting the polypeptide. Examples of chemical reagents are halogenating agents, particularly halogens of atomic no. 17-80. More particularly, iodine can be used under mild conditions and for sufficient time to achieve the desired results. Other suitable techniques include treatment with aldehydes, such as glutaraldehyde; anti-infectives, such as zephiran chloride and cetylpyridinium chloride; alcohols, such as isopropyl and ethanol; various histologic fixatives, such as Lugol iodine, Bouin's fixative, various acids and Helly's fixative (See: Humason, Gretchen L., Animal Tissue Techniques, W. H. Freeman and Company, 1967); or a combination of physical (heat) and chemical agents that preserve and prolong the activity of the polypeptide produced in the cell when the cell is administered to the host environment. Examples of physical means are short wavelength radiation such as gamma-radiation and X-radiation, freezing, UV irradiation, lyophilization, and the like. Methods for treatment of microbial cells are disclosed in U.S. Pat. Nos. 4,695,455 and 4,695,462, which are incorporated herein by reference.

The cells generally will have enhanced structural stability which will enhance resistance to environmental conditions. Where the pesticide is in a proform, the method of cell treatment should be selected so as not to inhibit processing of the proform to the mature form of the pesticide by the target pest pathogen. For example, formaldehyde will crosslink proteins and could inhibit processing of the proform of a polypeptide pesticide. The method of treatment should retain at least a substantial portion of the bio-availability or bioactivity of the polypeptide.

Characteristics of particular interest in selecting a host cell for purposes of production include ease of introducing the SMP #1-encoding polynucleotide into the host, availability of expression systems, efficiency of expression, stability of the pesticide in the host, and the presence of auxiliary genetic capabilities. Characteristics of interest for use as a pesticide rmicrocapsule include protective qualities for the pesticide, such as thick cell walls, pigmentation, and intracellular packaging or formation of inclusion bodies; survival in aqueous environments; lack of mammalian toxicity; attractiveness to pests for ingestion; ease of killing and fixing without damage to the toxin; and the like. Other considerations include ease of formulation and handling, economics, storage stability, and the like.

### Growth of Cells

The cellular host containing the polynucleotide(s) may be grown in any convenient nutrient medium, where the DNA construct provides a selective advantage, providing for a selective medium so that substantially all or all of the cells retain the polynucleotide. These cells may then be harvested in accordance with conventional ways. Alternatively, the cells can be treated prior to harvesting.

### Formulations

Formulated bait granules containing an attractant of the S. marcescens isolate or recombinant microbes comprising the gene obtainable from the S. marcescens isolate disclosed herein, can be applied to the soil. Formulated product can also be applied as a seed-coating or root treatment or total plant treatment at later stages of the crop cycle. Plant and soil treatments of S. marcescens cells may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include rheological agents, surfactants, emulsifiers, dispersants, or polymers.

As would be appreciated by a person skilled in the art, the pesticidal concentration will vary widely depending upon the nature of the particular formulation, particularly whether it is a concentrate or to be used directly. The pesticide will be present in at least 1% by weight and may be 100% by weight. The dry formulations will have from about 1-95% by weight of the pesticide while the liquid formulations will generally be from about 1-60% by weight of the solids in the liquid phase. The formulations will generally have from about 10² to about 10⁴ cells/mg. These formulations will be administered at about 50 mg (liquid or dry) to 1 kg or more per hectare.

The formulations can be applied to the environment of the pest, e.g., soil and foliage, by spraying, dusting, sprinkling, or the like.

The following examples serve better to illustrate the invention described herein are not intended to limit the invention in any way. Those skilled in this art will recognize that there are several different parameters which may be altered using routine experimentation and are intended to be within the scope of this invention.

### EXAMPLE 1

### Culturing the S. marcescens Isolate

A subculture of the S. marcescens isolate isolated from soil was maintained on tryptic soy agar slants and stored at 4° C. SMP #1 was purified from cultures of NRRL B-23112 grown in 50 ml of tryptic soy broth-5% skim milk (TSB-SM media) or glucose mineral salts media (1 g KH₂PO₄, 1 g K₂HPO₄, 1 g NaCl, 0.7 g MgSO₄ 7H₂O, 4 g (NH₄)₂SO₄, 0.5g sodium citrate, 10 g glucose, in 1 liter Milli-Q water) incubated at 30° C. in 250 ml flasks on a rotary shaker at 200 rpm with a 2 inch throw. A 0.1 ml sample of a 19 hour culture grown in TSB-SM media was used to inoculate the growth media.

### EXAMPLE 2

### Purification of the Protease

SMP #1 was purified from cultures of S. marcescens NRRL B-23112. Culture supernatants containing the SMP was prepared as described above. Cells were pelleted from a 48-h culture by centrifugation at 10 000 g for 20 minutes at 4° C. The culture supernatant was decanted, preserved with toluene (1 drop/50 ml supernatant), and stored at 4° C. until use. Ammonium sulfate was added to the culture supernatant (pH 7.5) in 10% increments to 80% saturation and stirred at 4° C. for 1 hour after each addition. The precipitate from the incremental addition was collected by centrifugation at 10 000 g for 20 minutes at 4° C. and the pellets were resuspended in 50 mM TRIS/HCL (pH 8). Fractions containing proteolytic activity (40% to 80% saturation) were pooled and subjected to fractional precipitation with acetone. Acetone was added slowly, with stirring, at 4° C. to the pooled ammonium sulfate fractions that retained proteolytic activity. The mixture was precipitated at 4° C. for 1 hour. The concentration of acetone was increased in 10% increments from 20%-80% final saturation. The precipitate resulting from each incremental addition was collected by centrifugation at 10 000 g for 20 minutes at 4° C. The pellets were resuspended in 20 mM TRIS/HCL (pH 8). Fractions containing proteolytic activity (40%-60% saturation) were pooled and dialyzed against 20 liters of 5 mM TRISIHCL pH 8, 10 μM MgCl₂ overnight at 4° C. with stirring using Spectrapor dialysis tubing with a molecular mass exclusion limit of 10 000 Da. A 5-ml sample of the dialysate was diluted to 50 ml with 2 ml 40% (w/v) ampholytes of pH 3-10 and 43 ml of water. This preparation was loaded into a Rotofor preparative isoelectric focusing cell (BioRad) and focused at 12 W constant power for 4 hours. Fractions were collected and the pH recorded. Aliquots of 0.2 ml from each fraction were loaded onto mini-spin columns of AG 501-X8 mixed-bed resin to remove ampholytes. The aliquots were analyzed by SDS-PAGE and assayed for proteolytic activity as described below. Active fractions were pooled and diluted to 50 ml with water, refocused in the isoelectric focusing cell, and the fractions treated as above. Fractions that retained proteolytic activity and had a single band on SDS-PAGE analysis were pooled. The dialysate was collected and centrifuged at 5000 g for 10 minutes at 4° C. to remove insoluble material, and aliquots were stored at −20° C.

### EXAMPLE 3

### Assay of Proteolytic Activity

Proteolytic activity was determined by casein digestion (Kunitz 1947). Substrate consisting of 0.75 ml 1.0% (w/v) casein in 100 mM TRIS/HCL, 1 mM MgCl₂ (pH 8) at 30° C. Protease samples (0.1 ml) were brought to 30° C., added to the substrate and incubated at 30° C. After 30 minutes, the reaction was quenched with 0.5ml 10% (w/v) trichloroacetic acid (TCA). After 15 minutes at 25° C., the quenched reaction mixture was centrifuged at 10 000 g for 10 minutes to pellet precipitated protein and the A₂₈₀ of the supernatant was determined. One unit of proteolytic activity was defined as the amount of enzyme that produced an increase of absorbance at 280 nM of 0.1 under the conditions of the assay.

### EXAMPLE 4

### Assay for Lytic Activity Against A. suum

Live A. suum were placed in 1 liter cotton stoppered flasks containing 250 ml of 0.1 mg/ml enzyme preparations. Enzyme preparations were tested individually and included SMP #1, serratiopeptidase (SMP #2; Sigma), papain (Sigma), ficin (Sigma), and control solutions. After 12 to 36 hours, the nematodes were observed for lytic effects or death. Lytic effects are defined as blistering or lesions of the cuticle and/or protrusions of internal viscera through the cuticle or body openings.

TABLE 3
Assay for Lytic Activity Against A. suum

| | Protein | | | | | |
|---|---|---|---|---|---|---|
| Sample | conc. | 2- | 12 hours | | 24 hours | |
| flask | (mg/ml) | mercaptoethanol | Death | Lysis | Death | Lysis |
| Ringers' | 0 | 0 | (−) | (−) | (−) | (−) |
| Ringers' | 0 | 10 mM | (−) | (−) | (−) | (−) |
| 48 h sup. | 0.7 | 10 mM | (+) | (++) | (+) | (+++) |
| 108 h sup. | 0.8 | 10 mM | (+) | (−) | (+) | (+) |
| SMP #1 | 0.1 | 0 | (−) | (−) | (−) | (−) |
| SMP #1 | 0.1 | 10 mM | (+) | (+++) | (+) | (+++) |
| SMP #2 | 0.1 | 0 | (−) | (−) | (−) | (−) |
| SMP #2 | 0.1 | 10 mM | (−) | (−) | (−) | (−) |
| Papain | 0.1 | 0 | (+) | (+++) | (+) | (+++) |
| Ficin | 0.1 | 0 | (−) | (−) | (−) | (−) |

(+) = degree of lysis;
(−) = no evidence of death or lysis

### EXAMPLE 5

### Isolations of A. suum Cuticle and Solubilization of Cuticle Components

Cuticle of adult A. suum was harvested (Fujimoto and Kanaya 1973). After several freeze-thaw cycles whole worms were mechanically stripped of their cuticle. The detached cuticles (14 g) were cut into about 1 cm to about 2 cm lengths and placed in 2L 0.5M NaCl solution, stirred at 4° C. for 24 hours with one change of buffer. The cuticle strips were collected by filtration and suspended in 100 ml 0.1M phosphate buffer pH 7, 1% (v/v) 2-mercaptoethanol. The suspension was stirred at 37° C. for 24 hours and the insoluble material collected by centrifugation at 10 000 g for 20 minutes at 25° C. The supernatant containing solubilized cuticular collagens was stored at 4° C. The pellet was collected, washed once with 50 ml of 0.1M phosphate buffer pH 7, 1% (v/v) 2-mercaptoethanol then twice with 100 ml Milli-Q water, suspended in 100 ml of Milli-Q water and stored at 4° C.

### EXAMPLE 6

### Assay Digestion of Cuticular Components

Soluble collagens and insoluble cuticulin harvested from A. suum were assayed for degradation by SMP #1 and serratiopeptidase (Sigma). Screening for degradation of A. suum collagens was done by placing 0.1 ml of solubilized collagens in 0.1 M phosphate buffer pH 7, 1% (v/v) 2-mercaptoethanol in a microcentrifuge tube as substrate. Enzyme preparations were added to the substrate as follows: SMP #1, 50 U in 50 μl. Milli-Q water (50 μl) was added to substrate as the control. The reaction mixtures were incubated at 37° C. and individual tubes removed for analysis every 12 hours for 84 hours. The reaction products were electrophoresed by SDS-PAGE (Shaggar and von Jagow 1987) and analyzed for disappearance of substrate collagens and appearance of low molecular weight degradation products. Cuticulin prepared from A. suum cuticle in Mill-Q water was centrifuged at 10 000 g for 20 minutes and the pellet resuspended in sterile Ringers solution containing 10 mM 2-mercaptoethanol, pH 6.8. The insoluble cuticulin fibers were homogenized with a Potter-Elvehjam tissue grinder. Microcentlifuge tubes containing 250 μl of the cuticulin homogenate were prepared and 50 μl of enzyme preparation added as previously described. The reaction mixtures were incubated at 37° C. and individual tubes removed for analysis every 12 hours for 84 hours. The collected reaction tubes were centrifuged at 10 000 g for 15 minutes to pellet the insoluble cuticulin. Cuticulin digest supernatant (250 μl) was precipitated with 1 ml of −20° C. acetone and placed at −20° C. for 24 hours. The precipitant was collected by centrifugation at 10 000 g for 20 minutes at 4° C., resuspended in 15 μl Milli-Q water, 15 μl sample buffer, and heated at 100° C. for 5 minutes. The samples were clectrophoresed on 12%(wt/vol) polyacrylamide gels (Schagger and von Jagow 1987) and stained with Coomassie blue.

### EXAMPLE 7

### Assay for Lytic Activity Against R. similis

The assays were performed in microtiter plates. Ten microliters of R. similissuspensions were placed in microtiter plate wells. Serially diluted SMP #1 (100U in 0.1 ml stock) was added to the wells. PBS was added to a series of wells containing R. similis as a control. The nematodes were stored at 20° C. and examined for lytic effects or death at 48 and 96 hours.

TABLE 4
Effect of SMP #1 and SMP #2 on R. similis

| | | | | | % |
|---|---|---|---|---|---|
| Treatment | U/well | Wells | Live | Dead | mortality^{a} |
| None | 0 | 10 | 233 | 67 | 22 |
| SMP #2 | 10 | 10 | 175 | 109 | 38 |
| SMP #1 | 10 | 10 | 2 | 298 | 99 |
| SMP #1 | 10 | 10 | 0 | 300 | 100 |

^{a}= significant difference (p < 0.05) between treatments

### EXAMPLE 8

### Inhibition of Protease Activity

Several compounds were tested as to characterize the novel protein. Effect of protease inhibitors (30-120 μ/ml antipain dihydrochloride, 1-4 μ/ml aprotinin, 20-80 μg/ml bestatin, 25-50 μg/ml chymostatin, 5-20 μg/ml E-64, 10-60 mM EDTA, 1-74 μg/ml leupeptin, 500-2000 μg/ml Pefabloc SC, 1-4 μ/ml pepstatin, 165-660 μlg/ml phosphoramidon and 100-400 μg/ml PMSF), SDS (0.1%-1%), Tween-20 (0.1%-1%), Triton X-100 (0.1%-1%), ethanol (1%-5%), and 2-mercaptoethanol (0.1%-1%) were determined by addition to the standard reaction mixture. Residual proteolytic activity was determined after incubation at 25° C. for 30 minutes.

### EXAMPLE 9

### Native Isoelectric Focusing

The isoelectric point (pI) of purified SMP#1 was determined using a vertical mini-gel system (Roberston et al. 1987) and 1.5 mm polyacrylamide gels consisting of 5% (w/v) 30:1 acrylamide/bisacrylamide, 10% (v/v) glycerol and 2% (w/v) pH 4-8 ampholytes. Electrode solutions were cooled to 4° C. prior to electrophoresis. The anode solution was 20 mM acetic acid and the cathode solution was 25 mM NaOH. Samples and protein standards (Sigma) were mixed with an equal volume of 50% (v/v) glycerol and 4%(w/v) ampholytes in the pH range of 4-8. Samples were prepared for isoelectric focusing by concentrating and desalting in a centriprep concentrator (Amicon) with a molecular mass exclusion limit of 10 000 Da. Electrophoresis was at 25° C. and 200 volts for 1.5 hours. The gels were fixed with 10% (w/v) trichloroacetic acid for 10 minutes with gentle agitation. Ampholytes were removed by shaking the gel for 2 hours in 1% (w/v) trichloroacetic acid. After rinsing with Milli-Q-water, the gels were stained with 0.25% (w/v) Coomassie blue R-250 in 45% (v/v) methanol and 10% (v/v) acetic acid for 10 minutes. The gels were destained in 45% (v/v) methanol and 10% (v/v) acetic acid until the background was clear.

### EXAMPLE 10

### Peptide Mapping

SMP #1 and serratiopeptidase were electrophoresed on 12% (w/v) polyacrylamide gels (Laemmli 1970) and stained briefly with 0.1% (w/v) Coomassie blue dye. After destaining, slices of 2 mm×5 mm that contained the bands corresponding to the proteases were excised from the gels. The gel slices were placed in microcentrifuge tubes with 0.5 ml extraction buffer (50 mM TRIS/HCL, 0.05% (w/v) SDS, 0.2 M sodium bicarbonate, 0.1 mM EDTA and macerated. The macerated gel slice preparations were placed at 4° C. for 12 hours and centrifuged at 10 000 g for 10 minutes to pellet the acrylamide. The supernatants containing the electrophoretically purified protease were collected and their purity verified by SDS-PAGE followed by silver staining (BioRad Silver Stain Plus Kit). Substrate samples of SMP #1 and serratiopeptidase (SMP #2) were prepared for digestion (Cleveland et al. 1977). The purified proteins were diluted to 0.5 mg/ml in buffer consisting of 125 mM TRIS/HCL pH 6.8, 0.5% (w/v) SDS, 10% (v/v) glycerol, and 0.001% (w/v) bromophenol blue. The samples were denatured by heating at 100° C. for 2 minutes. SMP 6.1 and serratiopeptidase were digested at 37° C. for 30 minutes by addition of alkaline protease (2.5, 5, 10 μg), Lys-C (0.1, 0.5, 1 μg), papain (0.5, 1, 2 μg), ficin 1.5, 3, 6 μg), trypsin 2.5, 5, 10 μg), and Glu-C (1, 2, 4 μg). the proteolytic digestion products were precipitated with nine volumes of ice-cold acetone and placed at −20° C. for 12 hours. The samples were centrifuged at 10 000 g for 15 minutes at 25° C. to pellet the precipitated digestion products. The acetone was removed and pellet dried at 37° C. The samples were resuspended in 15 μl water and 15 μl sample buffer and heated at 100° C. for 5 minutes before being loaded into a 1.5-mm 15% (w/v) acrylamide gel and electrophoresed (Schagger and von Jagow 1987). The gels were stained with Coomassie blue and silver stained.

### EXAMPLE 11

### PCR Cloning and Nucleotide Sequencing of Genomic DNA from Serratia marcescens NRRL B-23112

Genomic DNA from S. marcescens NRRL B-23112 was isolated by a standard CTAB (hexadecyltrimethylammonium bromide) procedure described below. The genomic DNA was used as a template in a PCR reaction with primers described below to selectively amplify the metalloproteinase gene and the specific inhibitor gene. Amplification and sequencing primers were designed using S. marcescens intergenic sequence data previously published in Genbank (Accession numbers X55521, X04127, L09107).

Isolation of Genomic DNA and PCR Cloning

Total cellular DNA was prepared from S. marcescens strain NRRL B-23112. A colony was inoculated to 5 ml LB and grown overnight at 37° C. 1.5 ml of the culture was microcentrifuged for 30 seconds at maximum speed. The pellet was resuspended in 567 μl TE buffer by repeated pipetting. 30 μl of 10%SDS and 3 μl of 20 mg/ml proteinase K, mix was added and incubate 1 hour at 37° C. Added 100 μl of 5M NaCl and mixed thoroughly. Then added 80 μl CTAB/NaCl solution (10% CTAB/0.7M NaCl), mixed and incubated 10 minutes at 65° C. An equal volume of chloroform/isoamyl alcohol was added then mixed, and microcentrifuged for 5 minutes at room temperature. The supernatant was transferred to a fresh tube and equal volumes of phenol/chloroform/isoamyl alcohol was added, mixed and microcentrifuged for 5 minutes. The supernatant was transferred to a fresh tube and 0.6 vol isopropanol was added and mixed until the DNA precipitated. The precipitate was transferred with a pipet tip in 1 ml of 70% ethanol and washed. Microcentrifuged precipitate for 5 minutes at room temperature and discarded supernatant and dried in lyophilizer for 5 minutes. The pellet was resuspended pellet in 100 μl TE buffer.

Employed PCR to obtain metalloprotease. PCR conditions were as follows:

| Mix 1: | 10 × PCR Buffer | 1× | |
|---|---|---|---|
| | MgCl₂ | 2.5 | mM |
| | DNTPs | 0.2 | mM each |
| | SMP S1 | 10 | pmol |
| | SMP AS2 | 10 | pmol |
| | double distilled | to 100 | μl |
| | H₂O | | |
| | Taq Polymerase | 5 | U |

| | |
|---|---|
| Template DNA | varies |

PCR was performed under the following conditions:

| | |
|---|---|
| Hotstart: | 98° C. for 2 minutes; total one cycle |
| Denature: | 45 seconds at 94° C. |
| Annealing: | 45 seconds at 58° C. |
| Extension: | 2 minutes at 72° C.; total 30 cycles; 10 minutes |
| | at 72° C.; total one cycle. |

Gene amplification primers for SMP #1:

| | |
|---|---|
| 5′GCTTACGGGGAGGTTATGTCTATCTC3′ | (SEQ ID |
| | NO: 12) |
| 5′GGTAGAGCTCGTGCGTGCTAAAGTACCTTTC3′ | (SEQ ID |
| | NO: 13) |

Sequencing primers for SMP #1:

| | |
|---|---|
| 5′GCTTACGGGGAGGTTATGTCTATCTC3′ | (SEQ ID |
| | NO: 14) |
| 5′GGTAGAGCTCGTGCGTGCTAAAGTACCTTTC3′ | (SEQ ID |
| | NO: 15) |
| 5′GGCCACTATGATTATGGTACCCA3′ | (SEQ ID |
| | NO: 16) |
| 5′GTTATACCGCTAACCAGCGC3′ | (SEQ ID |
| | NO: 17) |

Gene amplification and sequencing primers for SMP #1:

| | |
|---|---|
| 5′GTGAAAATCGTCGGCCAGGTAGACGTC3′ | (SEQ ID NO: 18) |
| 5′GGCTTTCTGTTACTTCTCCGGCCACTTC3′ | (SEQ ID NO: 19) |

Five microliters from each sample was analyzed in a 1% agarose gel. All these samples were combined and the PCR amplification products were purified by gel electrophoresis and used as template in PCR based cycle sequencing reactions using dideoxy fluorescent nucleotides to determine the nucleotide sequence.

### EXAMPLE 12

BLASTP alignment analyses detected significant homology (more than 90% overall amino acid sequence identity) between SMP #1 and SMP #2 (serratiopeptidase; Genbank accession no. X55521).

| | | |
|---|---|---|
| Query: | 1 | MQSTKKAIEITESSLAAATTGYDAVDDLLHYHERGNGIQINGKDSFSNEQAGLFITRENQ |
| 60 | | |
| Sbjct: | 7 | MQSTKKAIEITESSLAAATTGYDAVDDLLHYHERGNGIQINGKDSFSNEQAGLFITRENQ |
| 66 | | MQSTKKAIEITESSLAAATTCYDAVDDLLHYHERGNGIQINGKDSFSNEQAGLFITRENQ |
| Query: | 61 | TWNGYKVFGQPVKLTFSFPDYKFSSTNVAGDTGLSKFSAEQQQQAKLSLQSWADVANITF |
| 120 | | |
| Sbjct: | 67 | TWNGYKVFGQPVKLTFSFPDYKFSSTNVAGDTGLSKFSAEQQQQAKLSLQSWADVANITF |
| 126 | | TWNGYKVFGQPVKLTFSFPDYKFSSTNVAGDTGLSKFSAEQQQQAKLSLQSWADVANITF |
| Query: | 121 | TEVAAGQKANITFGNYSQDRPGHYDYGTQAYAFLPNTIWQGQDLGGQTWYNVNQSNVKHP |
| 180 | | |
| Sbjct: | 127 | TEVAAGQKANITFGNYSQDRPGHYDYGTQAYAFLPNTIWQGQDLGGQTWYNVNQSNVKHP |
| 186 | | TEVAAGQKANITFGNYSQDRPGHYDYGTQAYAFLPNTIWQGQDLGGQTWYNVNQSNVKHP |
| Query: | 181 | ATEDYGRQTFTHEIGHALGLSHPGDYNAGEGNPTYNDVTYAEDTRQFSLMSYWSETNTGG |
| 240 | | |
| Sbjct: | 187 | ATEDYGRQTFTHEIGHALGLSHPGDYNAGEGNPTYNDVTYAEDTRQFSLMSYWSETNTGG |
| 246 | | ATEDYGRQTFTHEIGHALGLSHPGDYNAGEGNPTYNDVTYAEDTRQFSLMSYWSETNTGG |
| Query: | 241 | DNGGHYXXXXXXXXXXXXQHLYGANLSTRTGDTVYGFNSNTGRDFLSTTSNSQKVIFAAW |
| 300 | | |
| Sbjct: | 247 | DNGGHY QHLYGAN STRTGDTVYGFNSNTGRDFLSTTSNSQKVIFAAW |
| 306 | | DNGGHYAAAPLLDDIAAIQHLYGANPSTRTGDTVYGFNSNTGRDFLSTTSNSQKVIFAAW |
| Query: | 301 | DAGGNDTFDFSGYTANQRINLNEKSFSDVGGLKGNVSIAAGVTIENAIGGSGNDVIVGNA |
| 360 | | |
| Sbjct: | 307 | DAGGNDTFDFSGYTANQRINLNEKSFSDVGGLKGNVSIAAGVTIENAIGGSGNDVIVGNA |
| 366 | | DAGGNDTFDFSGYTANQRINLNEKSFSDVGGLKGNVSIAAGVTIENAIGGSGNDVIVGNA |
| Query: | 361 | ANNVLKGGAGNDVLFXXXXXXXXXXXXXKDIFVFXXXXXXXXXXXXWIRDFQKGIDKIDL |
| 420 | | |
| Sbjct: | 367 | ANNVLKGGAGNDVLF KDIFVF WIRDFQKGIDKIDL |
| 426 | | ANNVLKGGAGNDVLFGGGGADELWGGAGKDIFVFSAASDSAPGASDWIRDFQKGIDKIDL |
| Query: | 421 | SFFNKEAQSSDFIHFVDHFSGTAGEALLSYNASSNVTDLSVNIGGHQAPDFLVKIVGQVD |
| 480 | | |
| Sbjct: | 427 | SFFNKEA SSDFIHFVDHFSGTAGEALLSYNASSNVTDLSVNIGGHQAPDFLVKIVGQVD |
| 486 | | SFFNKEANSSDFIHFVDHFSGTAGEALLSYNASSNVTDLSVNIGGHQAPDFLVKIVGQVD |
| Query: | 481 | VATDFIV 487 |
| | | VATDFIV |
| Sbjct: | 487 | VATDFIV 493 |

TABLE 5
Nucleotide Sequence Alignment of Selected SMPs

| | | |
|---|---|---|
| | | 1 30 |
| SMP #1 | (1) | ATGCAATCTACTAAAAAGGCAATTGAAATT |
| SMP #3 | (1) | ATGCAATCTACTAAAAAGGCAATTGAAATT |
| SMP #2 | (1) | ATGCAATCTACTAAAAAGGCAATTGAAATT |
| Consensus | (1) | ATGCAATCTACTAAAAAGGCAATTGAAATT |
| | | 31 60 |
| SMP #1 | (31) | ACTGAATCCAGCCTTGCGGCCGCGACAACC |
| SMP #3 | (31) | ACTGAATCCAACTTCGCGGCCGCCACAACC |
| SMP #2 | (31) | ACTGAATCCAGCCTTGCGGCCGCGACAACC |
| Consensus | (31) | ACTGAATCCAGCCTTGCGGCCGCGACAACC |
| | | 61 90 |
| SMP #1 | (61) | GGCTACGATGCTGTAGATGACCTGCTCCAT |
| SMP #3 | (61) | GGCTACGATGCTGTAGACGACCTGTTGCAT |
| SMP #2 | (61) | GGCTACGATGCTGTAGACGACCTGCTGCAT |
| Consensus | (61) | GGCTACGATGCTGTAGATGACCTGCTGCAT |
| | | 91 120 |
| SMP #1 | (91) | TATCATGAGCGGGGTAACGGGATTCAGATT |
| SMP #3 | (91) | TATCATGAGCGGGGCAACGGGATTCAGATT |
| SMP #2 | (91) | TATCATGAGCGGGGTAACGGGATTCAGATT |
| Consensus | (91) | TATCATGAGCGGGGTAACGGGATTCAGATT |
| | | 121 150 |
| SMP #1 | (121) | AATGGCAAGGATTCATTTTCTAACGAGCAA |
| SMP #3 | (121) | AATGGCAAGGATTCATTTTCTAACGAGCAA |
| SMP #2 | (121) | AATGGCAAGGATTCATTTTCTAACGAGCAA |
| Consensus | (121) | AATGGCAAGGATTCATTTTCTAACGAGCAA |
| | | 151 180 |
| SMP #1 | (151) | GCTGGGCTGTTTATTACCCGCGAGAACCAA |
| SMP #3 | (151) | GCTGGGCTGTTTATTACCCGTGACAACCAA |
| SMP #2 | (151) | GCTGGGCTGTTTATTACCCGCGAGAACCAA |
| Consensus | (151) | GCTGGGCTGTTTATTACCCGCGAGAACCAA |
| | | 181 210 |
| SMP #1 | (181) | ACCTGGAACGGTTACAAGGTATTTGGCCAG |
| SMP #3 | (181) | ACCTGGAACGGTTACAAGGTATTTGGCCAG |
| SMP #2 | (181) | ACCTGGAACGGTTACAAGGTATTTGGCCAG |
| Consensus | (181) | ACCTGGAACGGTTACAAGGTATTTGGCCAG |
| | | 211 240 |
| SMP #1 | (211) | CCGGTCAAATTAACCTTCTCCTTCCCGGAC |
| SMP #3 | (211) | CCGGTCAAATTAACCTTCTCCTTCCCGGAC |
| SMP #2 | (211) | CCGGTCAAATTAACCTTCTCCTTCCCGGAC |
| Consensus | (211) | CCGGTCAAATTAACCTTCTCCTTCCCGGAC |
| | | 241 270 |
| SMP #1 | (241) | TATAAGTTCTCTTCCACCAACGTCGCCGGC |
| SMP #3 | (241) | TATAAGTTCTCTTCCACCAACGTCGCCGGC |
| SMP #2 | (241) | TATAAGTTCTCTTCCACCAACGTCGCCGGC |
| Consensus | (241) | TATAAGTTCTCTTCCACCAACGTCGCCGGC |
| | | 271 300 |
| SMP #1 | (271) | GATACCGGGCTGAGCAAGTTCAGCGCGGAA |
| SMP #3 | (271) | GACACCGGGCTGAGCAAGTTCAGCGCGGAA |
| SMP #2 | (271) | GACACCGGGCTGAGCAAGTTCAGCGCGGAA |
| Consensus | (271) | GATACCGGGCTGAGCAAGTTCAGCGCGGAA |
| | | 301 330 |
| SMP #1 | (301) | CAGCAGCAGCAGGCTAAGCTGTCGCTGCAG |
| SMP #3 | (301) | CAGCAGCAGCAGGCTAAGCTGTCGCTGCAG |
| SMP #2 | (301) | CAGCAGCAGCAGGCTAAGCTGTCGCTGCAG |
| Consensus | (301) | CAGCAGCAGCAGGCTAAGCTGTCGCTGCAG |
| | | 331 360 |
| SMP #1 | (331) | TCCTGGGCCGACGTCGCCAATATCACCTTC |
| SMP #3 | (331) | TCCTGGGCCGACGTCGCCAATATCACCTTT |
| SMP #2 | (331) | TCCTGGGCCGACGTTGCCAATATCACCTTC |
| Consensus | (331) | TCCTGGGCCGACGTCGCCAATATCACCTTC |
| | | 361 390 |
| SMP #1 | (361) | ACCGAAGTGGCGGCCGGTCAAAAGGCCAAT |
| SMP #3 | (361) | ACCGAAGTGGCGGCCGGACAAAAGGCCAAC |
| SMP #2 | (361) | ACCGAAGTGGCGGCCGGTCAAAAGGCCAAT |
| Consensus | (361) | ACCGAAGTGGCGGCCGGTCAAAAGGCCAAT |
| | | 391 420 |
| SMP #1 | (391) | ATCACCTTCGGCAATTACAGCCAGGATCGT |
| SMP #3 | (391) | ATCACCTTCGGTAACTACAGCCAGGATCGT |
| SMP #2 | (391) | ATCACCTTCGGCAATTACAGCCAGGATCGT |
| Consensus | (391) | ATCACCTTCGGCAATTACAGCCAGGATCGT |
| | | 421 450 |
| SMP #1 | (421) | CCCGGCCACTATGATTATGGTACCCAGGCC |
| SMP #3 | (421) | CCCGGCCACTATGATTACGGCACCCAGGCC |
| SMP #2 | (421) | CCCGGCCACTATGATTATGGTACCCAGGCC |
| Consensus | (421) | CCCGGCCACTATGATTATGGTACCCAGGCC |
| | | 451 480 |
| SMP #1 | (451) | TACGCCTTCCTGCCGAACACCATTTGGCAG |
| SMP #3 | (451) | TACGCCTTCCTGCCGAACACCATTTGGCAG |
| SMP #2 | (451) | TACGCCTTCCTGCCGAACACCATTTGGCAG |
| Consensus | (451) | TACGCCTTCCTGCCGAACACCATTTGGCAG |
| | | 481 510 |
| SMP #1 | (481) | GGCCAGGATTTGGGCGGCCAGACCTGGTAC |
| SMP #3 | (481) | GGGCAGGATCTGGGGGGCCAGACCTGGTAC |
| SMP #2 | (481) | GGCCAGGATTTGGGCGGCCAGACCTGGTAT |
| Consensus | (481) | GGCCAGGATTTGGGCGGCCAGACCTGGTAC |
| | | 511 540 |
| SMP #1 | (511) | AACGTCAACCAATCCAACGTGAAGCATCCG |
| SMP #3 | (511) | AACGTCAACCAGTCCAACGTGAAGCATCCG |
| SMP #2 | (511) | AACGTCAACCAATCCAACGTGAAGCATCCG |
| Consensus | (511) | AACGTCAACCAATCCAACGTGAAGCATCCG |
| | | 541 570 |
| SMP #1 | (541) | GCGACCGAAGACTACGGCCGCCAGACGTTC |
| SMP #3 | (541) | GCGACCGAAGACTACGGCCGCCAGACGTTT |
| SMP #2 | (541) | GCGACCGAAGACTACGGCCGCCAGACGTTC |
| Consensus | (541) | GCGACCGAAGACTACGGCCGCCAGACGTTC |
| | | 571 600 |
| SMP #1 | (571) | ACCCATGAGATTGGCCATGCGCTGGGCCTG |
| SMP #3 | (571) | ACCCATGAGATTGGCCATGCGCTGGGTCTG |
| SMP #2 | (571) | ACCCATGAGATTGGCCATGCGCTGGGCCTG |
| Consensus | (571) | ACCCATCAGATTGGCCATGCGCTGGGCCTG |
| | | 601 630 |
| SMP #1 | (601) | AGCCACCCGGGCGACTACAACGCCGGTGAG |
| SMP #3 | (601) | AGCCATCCGGGCGATTACAACGCCGGTGAA |
| SMP #2 | (601) | AGCCACCCGGGCGACTACAACGCCGGTGAG |
| Consensus | (601) | AGCCACCCGGGCGACTACAACGCCGGTGAG |
| | | 631 660 |
| SMP #1 | (631) | GGCAACCCGACCTATAACGACGTTACCTAT |
| SMP #3 | (631) | GGCAACCCGACCTATCGCGACGTCACTTAT |
| SMP #2 | (631) | GGCAACCCGACCTATAACGACGTCACCTAT |
| Consensus | (631) | GGCAACCCGACCTATAACGACGTTACCTAT |
| | | 661 690 |
| SMP #1 | (661) | GCGGAAGATACCCGCCAGTTCAGCCTGATG |
| SMP #3 | (661) | GCGGAAGACACCCGTCAGTTCAGCCTGATG |
| SMP #2 | (661) | GCGGAAGATACCCGCCAGTTCAGCCTGATG |
| Consensus | (661) | GCGGAAGATACCCGCCAGTTCAGCCTGATG |
| | | 691 720 |
| SMP #1 | (691) | AGCTACTGGAGTGAAACCAACACCGGTGGC |
| SMP #3 | (691) | AGCTACTGGAGCGAAACCAACACCGGTGGT |
| SMP #2 | (691) | AGCTACTGGAGTGAAACCAACACCGGTGGC |
| Consensus | (691) | AGCTACTGGAGTGAAACCAACACCGGTGGC |
| | | 721 750 |
| SMP #1 | (721) | GACAACGGCGGTCACTATGCCGCGGCTCCG |
| SMP #3 | (721) | GATAACGGCGGTCATTACGCCGCAGCTCCG |
| SMP #2 | (721) | GACAACGGCGGTCACTATGCCGCGGCTCCG |
| Consensus | (721) | GACAACGGCGGTCACTATGCCGCGGCTCCG |
| | | 751 780 |
| SMP #1 | (751) | CTGCTGGATGACATTGCCGCCATTCAGCAT |
| SMP #3 | (751) | CTGCTGGATGACATTGCCGCCATTCAACAT |
| SMP #2 | (751) | TTGCTGGATGACATTGCCGCCATTCAGCAT |
| Consensus | (751) | CTGCTGGATGACATTGCCGCCATTCAGCAT |
| | | 781 810 |
| SMP #1 | (781) | CTGTATGGCGCGAACCTGTCGACCCGCACC |
| SMP #3 | (781) | CTGTATGGCGCCAACCTGTCGACCCGCACC |
| SMP #2 | (781) | CTGTATGGCGCCAACCCGTCGACCCGCACC |
| Consensus | (781) | CTGTATGGCGCGAACCTGTCGACCCGCACC |
| | | 811 840 |
| SMP #1 | (811) | GGCGACACCGTGTACGGCTTTAACTCCAAT |
| SMP #3 | (811) | GGCGACACCGTGTACGGTTTTAACTCCAAC |
| SMP #2 | (811) | GGCGACACCGTGTACGGCTTTAACTCCAAC |
| Consensus | (811) | GGCGACACCGTGTACGGCTTTAACTCCAAT |
| | | 841 870 |
| SMP #1 | (841) | ACCGGTCGTGACTTCCTCAGCACCACCAGC |
| SMP #3 | (841) | ACCGGTCGTGACTTCCTCAGCACCACCAGC |
| SMP #2 | (841) | ACCGGTCGTGACTTCCTCAGCACCACCAGC |
| Consensus | (841) | ACCGGTCGTGACTTCCTCAGCACCACCAGC |
| | | 871 900 |
| SMP #1 | (871) | AATTCGCAGAAAGTGATCTTTGCGGCCTGG |
| SMP #3 | (871) | AATTCGCAGAAAGTGATCTTTGCGGCCTGG |
| SMP #2 | (871) | AATTCGCAGAAAGTGATCTTTGCGGCCTGG |
| Consensus | (871) | AATTCGCAGAAAGTGATCTTTGCGGCCTGG |
| | | 901 930 |
| SMP #1 | (901) | GATGCGGGTGGCAACGATACCTTCGACTTC |
| SMP #3 | (901) | GATGCGGGTGGCAACGATACCTTCGACTTC |
| SMP #2 | (901) | GATGCGGGTGGCAACGATACCTTCGACTTC |
| Consensus | (901) | GATGCGGGTGGCAACGATACCTTCGACTTC |
| | | 931 960 |
| SMP #1 | (931) | TCCGGTTATACCGCTAACCAGCGCATCAAC |
| SMP #3 | (931) | TCCGGTTATACCGCTAACCAGCGCATCAAC |
| SMP #2 | (931) | TCCGGTTATACCGCTAACCAGCGCATCAAC |
| Consensus | (931) | TCCGGTTATACCGCTAACCAGCGCATCAAC |
| | | 961 990 |
| SMP #1 | (961) | CTGAATGAGAAATCGTTCTCCGACGTGGGC |
| SMP #3 | (961) | CTGAACGAGAAGTCGTTCTCCGACGTGGGC |
| SMP #2 | (961) | CTGAATGAGAAATCGTTCTCCGACGTGGGC |
| Consensus | (961) | CTGAATGAGAAATCGTTCTCCGACGTGGGC |
| | | 991 1020 |
| SMP #1 | (991) | GGCCTGAAGGGCAACGTCTCGATCGCCGCC |
| SMP #3 | (991) | GGCCTGAAAGGCAACGTCTCGATCGCCGCC |
| SMP #2 | (991) | GGCCTGAAGGGCAACGTCTCGATAGCCGCC |
| Consensus | (991) | GGCCTGAAGGGCAACGTCTCGATCGCCGCC |
| | | 1021 1050 |
| SMP #1 | (1021) | GGTGTGACCATTGAGAACGCCATTGGCGGT |
| SMP #3 | (1021) | GGTGTGACCATCGAGAACGCCATTGGC--T |
| SMP #2 | (1021) | GGTGTGACCATTGAGAACGCCATTGGCGGT |
| Consensus | (1021) | GGTGTGACCATTGAGAACGCCATTGGCGGT |
| | | 1051 1080 |
| SMP #1 | (1051) | TCCGGCAACGACGTGATCGTCGGCAACGCG |
| SMP #3 | (1049) | TCCGGCAACGAC-TGATCGTCGCCAACGCG |
| SMP #2 | (1051) | TCCGGCAATGACGTGATCGTCGGCAACGCG |
| Consensus | (1051) | TCCGGCAACGACGTGATCCTCGGCAACGCG |
| | | 1081 1110 |
| SMP #1 | (1081) | GCCAACAACGTGCTGAAAGGCGGCGCGGGT |
| SMP #3 | (1078) | GCCAATAACGTGCTGAAAGGCGGCGCGGGT |
| SMP #2 | (1081) | GCCAACAACGTGCTGAAAGGCGGCGCGGGT |
| Consensus | (1081) | CCCAACAACGTGCTGAAAGGCGGCGCGGGT |
| | | 1111 1140 |
| SMP #1 | (1111) | AACGACGTGCTGTTCGGCGGCGGCGGGGCG |
| SMP #3 | (1108) | AACGACGTGCTGTTCGGCGGCGGCGGGGCG |
| SMP #2 | (1111) | AACGACGTGCTGTTCGGCGGCGGCGGGGCG |
| Consensus | (1111) | AACGACGTGCTGTTCGGCGGCGGCGGGGCC |
| | | 1141 1170 |
| SMP #1 | (1141) | GATGAACTGTGGGGCGGTGCCGGCAAAGAC |
| SMP #3 | (1138) | GATGAGCTGTGGGGCGGTGCCGGTAAAGAC |
| SMP #2 | (1141) | GATGAACTGTGGGGCGGTGCCGGCAAAGAC |
| Consensus | (1141) | GATGAACTGTGGGGCGGTGCCGGCAAAGAC |
| | | 1171 1200 |
| SMP #1 | (1171) | ATCTTTGTGTTCTCTGCCGCCAGCGATTCC |
| SMP #3 | (1168) | ATCTTCGTGTTCTCTGCCGCCAGCGATTCC |
| SMP #2 | (1171) | ATCTTTGTGTTCTCTGCCCCCAGCGATTCC |
| Consensus | (1171) | ATCTTTGTGTTCTCTGCCGCCAGCGATTCC |
| | | 1201 1230 |
| SMP #1 | (1201) | GCACCGGGTGCTTCCGACTGGATCCGCGAC |
| SMP #3 | (1198) | GCGCCGGGCGCTTCAGACTGGATCCGCGAC |
| SMP #2 | (1201) | GCACCGGGTGCTTCCGACTGGATCCGCGAC |
| Consensus | (1201) | GCACCGGGTGCTTCCGACTGGATCCGCGAC |
| | | 1231 1260 |
| SMP #1 | (1231) | TTCCAGAAAGGGATCGACAAGATCGACCTG |
| SMP #3 | (1228) | TTCCAGAAAGGGATCGACAAGATTGATCTT |
| SMP #2 | (1231) | TTCCAGAAAGGGATCGACAAGATCGACCTG |
| Consensus | (1231) | TTCCAGAAAGGGATCGACAAGATCGACCTG |
| | | 1261 1290 |
| SMP #1 | (1261) | TCGTTCTTCAATAAAGAAGCGCAGAGCAGC |
| SMP #3 | (1258) | TCGTTCTTCAACAAAGAAGCGCAGAGCAGC |
| SMP #2 | (1261) | TCGTTCTTCAATAAAGAAGCGAATAGCAGT |
| Consensus | (1261) | TCGTTCTTCAATAAAGAAGCGCAGAGCACC |
| | | 1291 1320 |
| SMP #1 | (1291) | GATTTCATTCACTTCGTCGATCACTTCAGC |
| SMP #3 | (1288) | GATTTCATTCACTTCGTCGATCACTTCAGC |
| SMP #2 | (1291) | GATTTCATCCACTTCGTCGATCACTTCAGC |
| Consensus | (1291) | GATTTCATTCACTTCGTCGATCACTTCAGC |
| | | 1321 1350 |
| SMP #1 | (1321) | GGCACGGCCGGTGAGGCGCTGCTGAGCTAC |
| SMP #3 | (1318) | GGCGCGGCCGGTGAAGCGCTGCTGAGCTAC |
| SMP #2 | (1321) | GGCACGGCCGGTGAGGCGCTGCTGAGCTAC |
| Consensus | (1321) | GGCACGGCCGGTGAGGCGCTGCTGAGCTAC |
| | | 1351 1380 |
| SMP #1 | (1351) | AACGCGTCCAGCAACGTGACCGATTTGTCG |
| SMP #3 | (1348) | AACGCGTCCAACAACGTGACCGATTTGTCC |
| SMP #2 | (1351) | AACGCGTCCAGCAACGTGACCGATTTGTCG |
| Consensus | (1351) | AACGCGTCCAGCAACGTGACCGATTTGTCG |
| | | 1381 1410 |
| SMP #1 | (1381) | GTGAACATCGGCGGGCATCAGGCGCCGGAC |
| SMP #3 | (1378) | GTGAACATCGGTGGTCATCAGGCGCCTGAC |
| SMP #2 | (1381) | GTGAACATCGGCGGGCATCAGGCGCCGGAC |
| Consensus | (1381) | GTGAACATCGGCGGGCATCAGGCGCCGGAC |
| | | 1411 1440 |
| SMP #1 | (1411) | TTCCTGGTGAAAATCGTCGGCCAGGTAGAC |
| SMP #3 | (1408) | TTCCTGGTGAAAATCGTCGGTCAGGTAGAC |
| SMP #2 | (1411) | TTCCTGGTGAAAATCGTCGGCCAGGTAGAC |
| Consensus | (1411) | TTCCTGGTGAAAATCGTCGGCCAGGTAGAC |
| | | 1441 1464 |
| SMP #1 | (1441) | GTCGCCACGGACTTTATCGTGTAA |
| SMP #3 | (1438) | GTCGCCACTGACTTTATCGTGTAA |
| SMP #2 | (1441) | GTCGCCACGGACTTTATCGTGTAA |
| Consensus | (1441) | GTCGCCACGGACTTTATCGTGTAA |

SMP #1 = SEQ ID NO:1
SMP #2 = Genbank accession no. X55521 (SEQ ID NO:5)
SMP #3 = Genbank accession no. X04127 (SEQ ID NO:6)

TABLE 6
Amino Acid Sequence Alignment of Selected SMPs

| | | |
|---|---|---|
| | | 1 30 |
| SMP #1 | (1) | mqstkkaieitesslaaattgydavddllh |
| SMP #3 | (1) | mqstkkaieitesnfaaattgydavddllh |
| SMP #2 | (1) | mqstkkaieitesslaaattgydavddllh |
| | | 31 60 |
| SMP #1 | (31) | yhergngiqingkdsfsneqaglfitrenq |
| SMP #3 | (31) | yhergngiqingkdsfsneqaglfitrenq |
| SMP #2 | (31) | yhergngiqingkdsfsneqaglfitrenq |
| | | 61 90 |
| SMP #1 | (61) | twngykvfgqpvkltfsfpdykfsstnvag |
| SMP #3 | (61) | twngykvfgqpvkltfsfpdykfsstnvag |
| SMP #2 | (61) | twngykvfgqpvkltfsfpdykfsstnvag |
| | | 91 120 |
| SMP #1 | (91) | dtglskfsaeqqqqaklslqswadvanitf |
| SMP #3 | (91) | dtglskfsaeqqqqaklslqswadvanitf |
| SMP #2 | (91) | dtglskfsaeqqqqaklslqswadvanitf |
| | | 121 150 |
| SMP #1 | (121) | tevaagqkanitfgnysqdrpghydygtqa |
| SMP #3 | (121) | tevaagqkanitfgnysqdrpghydygtqa |
| SMP #2 | (121) | tevaagqkanitfgnysqdrpghydygtqa |
| | | 151 180 |
| SMP #1 | (151) | yaflpntiwqgqdlggqtwynvnqsnvkhp |
| SMP #3 | (151) | yaflpntiwqgqdlggqtwynvnqsnvkhp |
| SMP #2 | (151) | yaflpntiwqgqdlggqtwynvnqsnvkhp |
| | | 181 210 |
| SMP #1 | (181) | atedygrqtftheighalglshpgdynage |
| SMP #3 | (181) | atedygrqtftheighalglshpgdynage |
| SMP #2 | (181) | atedygrqtftheighalglshpgdynage |
| | | 211 240 |
| SMP #1 | (211) | gnptyndvtyaedtrqfslmsywsetntgg |
| SMP #3 | (211) | gnptyrdvtyaedtrqfslmsywsetntgg |
| SMP #2 | (211) | gnptyndvtyaedtrqfslmsywsetntgg |
| | | 241 270 |
| SMP #1 | (241) | dngghyaaapllddiaaiqhlyganlstrt |
| SMP #3 | (241) | dngghyaaapllddiaaiqhlyganlstrt |
| SMP #2 | (241) | dngghyaaapllddiaaiqhlyganpstrt |
| | | 271 300 |
| SMP #1 | (271) | gdtvygfnsntgrdflsttsnsqkvifaaw |
| SMP #3 | (271) | gdtvygfnsntgrdflsttsnsqkvifaaw |
| SMP #2 | (271) | gdtvygfnsntgrdflsttsflsqkvifaaw |
| | | 301 330 |
| SMP #1 | (301) | daggndtfdfsgytanqrinlneksfsdvg |
| SMP #3 | (301) | daggndtfdfsgytanqrinlneksfsdvg |
| SMP #2 | (301) | daggndtfdfsgytanqrinlneksfsdvg |
| | | 331 360 |
| SMP #1 | (331) | glkgnvsiaagvtienaiggsgndvivgna |
| SMP #3 | (331) | glkgnvsiaagvtienaig-frqrlivgna |
| SMP #2 | (331) | glkgnvsiaagvtienaiggsgndvivgna |
| | | 361 390 |
| SMP #1 | (361) | annvlkggagndvlfggggadelwggagkd |
| SMP #3 | (361) | annvlkggagndvlfggggadelwggagkd |
| SMP #2 | (361) | annvlkggagndvlfggggadelwggagkd |
| | | 391 420 |
| SMP #1 | (391) | ifvfsaasdsapgasdwirdfqkgidkidl |
| SMP #3 | (391) | ifvfsaasdsapgasdwirdfqkgidkidl |
| SMP #2 | (391) | ifvfsaasdsapgasdwirdfqkgidkidl |
| | | 421 450 |
| SMP #1 | (421) | sffnkeaqssdfihfvdhfsgtageallsy |
| SMP #3 | (421) | sffnkeaqssdfihfvdhfsgaageallsy |
| SMP #2 | (421) | sffnkeanssdfihfvdhfsgtageallsy |
| | | 451 480 |
| SMP #1 | (451) | nassnvtdlsvnigghqapdflvkivgqvd |
| SMP #3 | (451) | nasnnvtdlsvnigghqapdflvkivgqvd |
| SMP #2 | (451) | nassnvtdlsvnigghqapdflvkivgqvd |
| | | 481 503 |
| SMP #1 | (481) | vatdfiv* |
| SMP #3 | (481) | vatdfiv* |
| SMP #2 | (481) | vatdfiv* |

SMP #1 = SEQ ID NO:2
SMP #2 = Genbank accession no. X55521 (SEQ ID NO:7)
SMP #3 = Genbank accession no. X04127 (SEQ ID NO:8)

TABLE 7

Nucleotide Sequence
Alignment of Selected SmaPIs

| | | |
|---|---|---|
| | | 1 30 |
| SmaPI #1 | (1) | ATGAAAGGTACTTTAGCGCGCACCGCTTTG |
| SmaPI #2 | (1) | ATGAAAGGTACTTTAGCACGCACCGCTTTG |
| SmaPI #3 | (1) | ATGAAAGGTACTTTAACGCGCGCCGCCCTG |
| Consensus | (1) | ATGAAAGGTACTTTAGCGCGCACCGCTTTG |
| | | 31 60 |
| SmaPI #1 | (31) | GCGGCGGGTGGCATGATGGTGACGAGTGCG |
| SmaPI #2 | (31) | GCGGCGGGTGGCATGATGGTGACGAGTGCG |
| SmaPI #3 | (31) | GCGGCGGGAGGCATGATGGTGACGAGTGCG |
| Consensus | (31) | GCGGCGGGTGGCATGATGGTGACGAGTGCG |
| | | 61 90 |
| SmaPI #1 | (61) | GTGATGCCCGGCAGTTTGGCATTGCCGACC |
| SmaPI #2 | (61) | GTGATGGCCGGCAGTTTGGCATTGCCGACC |
| SmaPI #3 | (61) | GTGATGGCCGGCAGTCTGGCGCTGCCGACC |
| Consensus | (61) | GTGATGGCCGGCAGTTTGGCATTGCCGACC |
| | | 91 120 |
| SmaPI #1 | (91) | GCGCAGTCGCTGGCGGGGCAATGGCAGGTG |
| SmaPI #2 | (91) | GCGCAGTCGCTGGCGGGACAATGGCAGGTG |
| SmaPI #3 | (91) | GCGCAGTCGCTGGCGGGGCAATGGGAGGTA |
| Consensus | (91) | GCGCAGTCGCTGGCGGGGCAATGGCAGGTG |
| | | 121 150 |
| SmaPI #1 | (121) | GCCGACAGCGAACGGCAATGCCAAATCGAG |
| SmaPI #2 | (121) | GCCGACAGCGAACGGCAATGCCAAATCGAG |
| SmaPI #3 | (121) | GCCGACAGCGAACGGCAATGCCAAATCGAG |
| Consensus | (121) | GCCGACAGCGAACGGCAATGCCAAATCGAG |
| | | 151 180 |
| SmaPI #1 | (151) | TTTCTGGCGCATGAACAAAACGAGACCAAC |
| SmaPI #2 | (151) | TTTCTGGCGCATGAACAAAGCGAGACCAAC |
| SmaPI #3 | (151) | TTTCTGGCCAATGAGCAAAGCGAGACCAAC |
| Consensus | (151) | TTTCTGGCGCATGAACAAAGCGAGACCAAC |
| | | 181 210 |
| SmaPI #1 | (181) | GGCTATCAGCTGGTGGATCGGCAACAGTGT |
| SmaPI #2 | (181) | GGCTATCAGCTGGTGGATCGGCAACAGTGT |
| SmaPI #3 | (181) | GGCTACCAGCTGGTGGATCGGCAGCGTTGT |
| Consensus | (181) | GGCTATCAGCTGGTGGATCGGCAACAGTGT |
| | | 211 240 |
| SmaPI #1 | (211) | TTGCAGAGCGTGTTTGCGGCGGAAGTCGTG |
| SmaPI #2 | (211) | TTGCAGAGCGTGTTTGCGGCGGAAGTCGTG |
| SmaPI #3 | (211) | CTGCAAAGCGTATTTGCGGCCGAGGTGGTG |
| Consensus | (211) | TTGCAGAGCGTGTTTGCGGCGGAAGTCGTG |
| | | 241 270 |
| SmaPI #1 | (241) | GGCTGGCGCCCGGCTCCGGACGGCATCGCC |
| SmaPI #2 | (241) | GGCTGGCGCCCGGCTCCGGACGGCATCGCC |
| SmaPI #3 | (241) | G-CTGGCGC--GGGGCCGGACGGCATCGCC |
| Consensus | (241) | GGCTGGCGCCCGGCTCCGGACGGCATCGCC |
| | | 271 300 |
| SmaPI #1 | (271) | TTGCTGCGGGCGGATGGCAGCACGCTGGCG |
| SmaPI #2 | (271) | CTGCTGCGGGCGGATGGCAGCACGCTGGCG |
| SmaPI #3 | (268) | CTGCTGCAGGCGGATGGCAGCACGCTGGCG |
| Consensus | (271) | CTGCTGCGGGCGGATGGCAGCACGCTGGCG |
| | | 301 330 |
| SmaPI #1 | (301) | TTCTTCTCGCGCGACGGCGATCTGTACCGC |
| SmaPI #2 | (301) | TTCTTCTCGCGCGA-GGC-ATCTGTACCGC |
| SmaPI #3 | (298) | TTCTTCTCGCGCGACGGCGATCTATACCGC |
| Consensus | (301) | TTCTTCTCGCGCGACGGCGATCTGTACCGC |
| | | 331 360 |
| SmaPI #1 | (331) | AATCAGCTGGGTGCGGATGATGCTCTGACG |
| SmaPI #2 | (329) | AATCAGCTGGGTGCGGATGATGCTTTGACG |
| SmaPI #3 | (328) | AATCAGCTGGGTGCGGGTGATGCCCTGACG |
| Consensus | (331) | AATCAGCTGGGTGCGGATGATGCTCTGACG |
| | | 361 381 |
| SmaPI #1 | (361) | TTGAAAGCGCTGGCTTGATGA |
| SmaPI #2 | (359) | TTGAAAGCGCTGGCTTGATGA |
| SmaPI #3 | (358) | TTGAAAGCGCTGGCTTGATGA |
| Consensus | (361) | TTGAAAGCGCTGGCTTGATGA |

SmaPI #1 = SEQ ID NO:3
SmaPI #2 = Genbank accession no. X55521 (SEQ ID NO:9)
SmaPI #3 = Genbank accession no. L09107 (SEQ ID NO:10)

TABLE 8

Amino Acid Sequence
Alignment of Selected SmaPIs

| | | |
|---|---|---|
| +TL,3 | | 1 25 |
| SmaPI #1 | (1) | mkgtlartalaaggmmvtsavmags |
| SmaPI #3 | (1) | mkgtltraalaaggmmvtsavmags |
| | | 26 50 |
| SmaPI #1 | (26) | lalptaqslagqwqvadserqcqie |
| SmaPI #3 | (26) | lalptaqslagqwevadserqcqie |
| | | 51 75 |
| SmaPI #1 | (51) | flaheqnetngyqlvdrqqclqsvf |
| SmaPI #3 | (51) | flaneqsetngyqlvdrqrclqsvf |
| | | 76 100 |
| SmaPI #1 | (76) | aaevvgwrpapdgiallradgstla |
| SmaPI #3 | (76) | aaevv-agagpdgiallqadgstla |
| | | 101 125 |
| SmaPI #1 | (101) | ffsrdgdlyrnqlgaddaltlkala |
| SmaPI #3 | (101) | ffsrdgdlyrnqlgagdaltlkala |

Sm aPI #1 = SEQ ID NO:4
SmaPI #3 = Genbank accession no. L09107 (SEQ ID NO:11)

It should be understood that the examples and embodiments described herein are for illustrative purposes only and that various modifications or changes in light thereof will be suggested to persons skilled in the art and are to be included within the spirit and purview of this application and scope of the appended claims.

The publications and other material used herein to illuminate the background of the invention or provide additional details respecting the practice, are herein incorporated by reference in their entirety.

### BIBLIOGRAPHY

Agrios, G. N. (1978) In: Plant Pathology. New York, Academic Press Inc. pp. 612-660.

Amado, R., Aeschbach, R., Neukom, H. (1984) Dityrosine: in vitro production and characterization. Methods Enzymol 107: 377-388.

Betschart B., Wyss, K. (1990) Analysis of the cuticular collagens of Ascaris suum. Acta trop 47: 297-305.

Fetterer R. H., Rhodes, M. L. (1993) Biochemistry of the nematode cuticle: relevance to parasitic nematodes of livestock. Vet Parasitol 46: 103-111.

Fry, S. C. (1987) Formation of isodityrosine by peroxidase isozymes. J. Exp Bot 38: 853-862.

Fujimoto, D. (1975) Action of bacterial collagenase on Ascaris cuticle collagen. J Biochem Tokyo 78: 9.5-909.

Fujimoto D., Kanaya, S. (1973) Cuticulin: a noncollagen structural protein from Ascaris cuticle. Arch Biochem Biophys 157: 1-6.

Humason, Gretchen L., (1967) Animal Tissue Techniques, W. H. Freeman and Company.

Johnstone, I. L. (1994) The cuticle of the nematode Caenorhabditis elegans: a complex collagen structure. Bioessays 16: 171-178.

Keller, G. H., M. M. Manak (1987) DNA Probes, Stockton Press, New York, N.Y., pp. 169-170

Kingston, I. B., Pettitt, J. (1990) Structure and expression of Ascaris suum collagen genes: a comparison with Caenorhabditis elegans. Acta Trop 47: 283-287.

Maniatis et al. (1982) Molecular Cloning: A Laboratory Manual, Cold Spring Harbor Laboratory, New York.

Peeters, H. (1976) In: Protides of the biological fluids. Pergammon Press, New York.

Salamone, P. R. and Wodzinski, R. J. (1997) Production, purification and characterization of a 50-kDa extracellular metalloprotease from Serratia marcescens. Appl Microbiol Biotechnol 48: 317-324.

Saiki et al. et al. (1985) Enzymatic Amplification of β-Globin Genomic Sequences and Restriction Site Analysis for Diagnosis of Sickle Cell Anemia. Science 230:1350-1354.

Shamansky, L. M., Pratt, D., Boisvenue, R. J., Cox, G. N. (1989) Cuticle collagen genes of Haemonchus contortus and Caenorhabditis elegans are highly conserved. Mol Biochem Parasitol 37: 73-85.

International Application No. WO93/16094

U.S. Pat. No. 4,695,455

U.S. Pat. No. 4,695,462

U.S. Pat. No. 5,380,831

U.S. Pat. No. 5,135,867

U.S. Pat. No. 5,011,909

U.S. Pat. No. 5,130,253

U.S. Pat. No. 4,683,195

U.S. Pat. No. 4,683,202

U.S. Pat. No. 4,800,159

### Sequence Listing

What is claimed is:

1. An isolated nucleic acid molecule comprising a polynucleotide selected from the group consisting of:
(a) a polynucleotide having 70% sequence identity to SEQ ID NO: 1;
(b) a polynucleotide having 80% sequence identity to SEQ ID NO: 1;
(c) a polynucleotide having 90% sequence identity to SEQ ID NO:1;
(d) a polynucleotide having 95% sequence identity to SEQ ID NO:1;
(e) a polynucleotide having the sequence of SEQ ID NO:1;
(f) a polynucleotide encoding a polypeptide having 70% sequence identity to SEQ ID NO:2;
(g) a polynucleotide encoding a polypeptide having 80% sequence identity to SEQ ID NO:2;
(h) a polynucleotide encoding a polypeptide having 90% sequence identity to SEQ ID NO:2;
(i) a polynucleotide encoding a polypeptide having 95% sequence identity to SEQ ID NO:2;
(j) a polynucleotide encoding the polypeptide of SEQ ID NO:2;
(k) a polynucleotide that hybridizes under conditions of high stringency to the polynucleotide of SEQ ID NO:1; and
(l) a polynucleotide complementary to a polynucleotide of (a) through (k).

2. A recombinant expression cassette comprising the isolated nucleic acid molecule of claim 1.

3. A vector comprising the recombinant expression cassette of claim 2.

4. A host cell comprising the vector of claim 3.

5. The host cell of claim 4, wherein the host cell is a plant cell.

6. The host cell of claim 5, wherein the plant cell is that of a plant capable of being affected by nematodes.

7. The host cell of claim 6, wherein the plant capable of being affected by nematodes is selected from the group consisting of maize, sorghum, wheat, alfalfa, sunflower, canola, cotton, rice, tomato, soybean, banana, citrus, and avocado.

8. A transformed plant comprising the isolated nucleic acid molecule of claim 1.

9. A plant seed comprising the isolated nucleic acid molecule of claim 1.

10. A method for making a serratia metalloprotease (SMP) comprising:
(a) expressing the isolated nucleic acid molecule of claim 1 in a recombinantly engineered cell, wherein the isolated nucleic acid molecule is operably linked to a promoter; and
(b) obtaining the protein.

11. An isolated polypeptide comprising a polypeptide selected from the group consisting of:
(a) a polypeptide having an amino acid sequence 70% identical to the amino acid sequence of SEQ ID NO:2;
(b) a polypeptide having an amino acid sequence 80% identical to the amino acid sequence of SEQ ID NO:2;
(c) a polypeptide having an amino acid sequence 90% identical to the amino acid sequence of SEQ ID NO:2;
(d) a polypeptide having an amino acid sequence 95% identical to the amino acid sequence of SEQ ID NO:2;
(e) a polypeptide having the amino acid sequence of SEQ ID NO:2; and
(f) a polypeptide which is encoded by the polynucleotide of SEQ ID NO:1.

12. A method for controlling a pest comprising administering to the pest a polypeptide having the amino acid sequence of SEQ ID NO:2.

13. The method of claim 12, wherein the pest is a nematode belonging to the genera selected from the group consisting of Ascaris and Radopholus.

14. A method for conferring nematode resistance to a plant comprising stably transforming the plant with an expression construct comprising a plant expression promoter operably linked to a polynucleotide encoding a polypeptide obtained from Serratia marcescens NRRL B-23112, wherein the polypeptide has specific nematocidal activity.

15. The method of claim 14, wherein the polynucleotide comprises SEQ ID NO: 1.

16. The method of claim 14, wherein the polypeptide has the amino acid sequence set forth in SEQ ID NO: 2.

17. The method of claim 14, wherein the plant is selected from the group consisting of maize, sorghum, wheat, alfalfa, sunflower, canola, cotton, rice, tomato, soybean, banana, citrus, and avocado.

18. A nematocidal protein isolated from a substantially purified culture of Serratia marcescens NRRL B-23112, and mutants thereof.

19. A polynucleotide encoding a nematocidal protein active against nematodes of the genera Radopholus and Ascaris, wherein the polynucleotide conserves the differences between SEQ ID NO:1 and SEQ ID NO:6, when compared using a BLAST algorithm.

20. An antibody which selectively binds to the polypeptide of claim 11.

21. An isolated nucleic acid molecule comprising a polynucleotide selected from the group consisting of:
(a) a polynucleotide having 70% sequence identity to SEQ ID NO: 3;
(b) a polynucleotide having 80% sequence identity to SEQ ID NO: 3;
(c) a polynucleotide having 90% sequence identity to SEQ ID NO:3;
(d) a polynucleotide having 95% sequence identity to SEQ ID NO:3;
(e) a polynucleotide having the sequence of SEQ ID NO:3;
(f) a polynucleotide encoding a polypeptide having 70% sequence identity to SEQ ID NO:4;
(g) a polynucleotide encoding a polypeptide having 80% sequence identity to SEQ ID NO:4;
(h) a polynucleotide encoding a polypeptide having 90% sequence identity to SEQ ID NO:4;
(i) a polynucleotide encoding a polypeptide having 95% sequence identity to SEQ ID NO:4;
(j) a polynucleotide encoding the polypeptide of SEQ ID NO:4;
(k) a polynucleotide that hybridizes under conditions of high stringency to the polynucleotide of SEQ ID NO:3; and
(l) a polynucleotide complementary to a polynucleotide of (a) through (k).

22. A recombinant expression cassette comprising the isolated nucleic acid molecule of claim 21.

23. A vector comprising the recombinant expression cassette of claim 22.

24. A host cell comprising the vector of claim 23.

25. The host cell of claim 24, wherein the host cell is a plant cell.

26. The host cell of claim 25, wherein the plant cell is that of a plant capable of being affected by nematodes.

27. The host cell of claim 26, wherein the plant capable of being affected by nematodes is selected from the group consisting of maize, sorghum, wheat, alfalfa, sunflower, canola, cotton, rice, tomato, soybean, banana, citrus, and avocado.

28. A transformed plant comprising the isolated nucleic acid molecule of claim 21.

29. A plant seed comprising the isolated nucleic acid molecule of claim 21.

30. A method for making a serratia metalloprotease inhibitor (SmaPI) comprising:
(a) expressing the isolated nucleic acid molecule of claim 21 in a recombinantly engineered cell, wherein the isolated nucleic acid molecule is operably linked to a promoter; and
(b) obtaining the protein.

31. An isolated polypeptide comprising a polypeptide selected from the group consisting of:
(a) a polypeptide having an amino acid sequence 70% identical to the amino acid sequence of SEQ ID NO:4;
(b) a polypeptide having an amino acid sequence 80% identical to the amino acid sequence of SEQ ID NO:4;
(c) a polypeptide having an amino acid sequence 90% identical to the amino acid sequence of SEQ ID NO:4;
(d) a polypeptide having an amino acid sequence 95% identical to the amino acid sequence of SEQ ID NO:4;
(e) a polypeptide having the amino acid sequence of SEQ ID NO:4; and
(f) a polypeptide which is encoded by the polynucleotide of SEQ ID NO:3.

32. A method for conferring nematode resistance to a plant comprising stably transforming the plant with an expression construct comprising the polynucleotide of SEQ ID NO:1 under the control of a promoter operably linked thereto.

33. The method of claim 32, wherein the plant is selected from the group consisting of maize, sorghum, wheat, alfalfa, sunflower, canola, cotton, rice, tomato, soybean, banana, citrus, and avocado.

34. The method of claim 32, wherein the expression construct further comprises the polynucleotide of SEQ ID NO:3.

35. The method of claim 34, wherein the polynucleotide of SEQ ID NO:1 and 3 are under the control of the same promoter.

36. An antibody which selectively binds to the polypeptide of claim 31.
